(19) United States
(12) Patent Application Publication
(10) Pub. No.: US 2012/0180150 A1
(43) Pub. Date: Jul. 12, 2012

(54) INOSITOL 1,4,5-TRIPHOSPHATE RECEPTOR-BINDING PROTEIN AND ITS NON-HUMAN TRANSGENIC MAMMALIAN ANIMALS

(76) Inventors: Takahiro Fujimoto, Fukuoka-shi (JP); Senji Shirasawa, Fukuoka-shi (JP)

(21) Appl. No.: 13/496,314

(22) PCT Filed: Sep. 28, 2010
(86) PCT No.: PCT/JP2010/066864

§ 371 Date: Mar. 15, 2012

(30) Foreign Application Priority Data
Sep. 29, 2009 (JP) 2009-225596

(51) Int. Cl. A01K 67/027; G01N 33/566; C12N 15/63; C07K 19/00

(52) U.S. Cl. 800/14; 530/350; 436/501; 435/320.1

(57) ABSTRACT

The instant invention provides an inositol 1,4,5-triphaosphate receptor (IP3R)-binding protein and its transgenic non-human mammalian animal.

The inositol 1,4,5-triphaosphate receptor-binding protein comprises an inositol 1,4,5-triphaosphate receptor-binding protein with a protein including KRAP protein (SQ ID NOS. 1-4) bound to inositol 1,4,5-triphosphate (IP3R) protein (SQ ID NOS. 5-8). The IP3R activity inhibitor contains the IP3R-binding protein as a major ingredient. The transgenic non-human mammal of the instant invention carries the IP3R-binding protein. The site of binding of the KRAP protein to the IP3R protein is located in an amino acid region composed of 19 amino acids ranging from amino acid 200 to amino acid 218 of the mouse KRAP protein and an 19-amino acid region corresponding to that of the mouse KRAP protein for any animal species other than mouse.

### TECHNICAL FIELD

The instant invention relates to an inositol 1,4,5-triphosphate receptor-binding protein and its non-human transgenic mammalian animals.

### BACKGROUND TECHNOLOGY

KRAP (Ki-ras-induced actin-interacting protein) gene coding for KRAP protein is identified by the present inventors from the cDNA library of human colon cancer cell line HCT116 as a gene of which the expression level can be up-regulated by activated Ki-ras (Non-patent literature document No. 1). The structure of the KRAP gene is conserved over a wide range crossing species of animals, i.e., from fish species to mammalian species. The expression pattern and molecular functions, however, are not yet clarified.

As a result of review on the histological expression of the KRAP protein in mouse tissue using a polyclonal antibody specific to the KRAP protein, the present inventors have found and reported that the KRAP protein demonstrates a ubiquitous expression in mouse physiological tissue and its expression level is high in the pancreas, liver and brown adipose tissue as well as it is present together with filamentous actin along the apical membrane part in the pancreas and liver tissue (Non-patent literature document No. 2). Studies of the KRAP protein subfractions have revealed that the KRAP protein is a cytosolic protein and its majority is associated with the cytoskeleton. The profiling of genetic expressions using a microarray while suppressing the KRAP expression in the colon cancer cell line HCT116 has also revealed that several kinds of receptors and signal molecules which are often deregulated in cancer cells cause a larger variation in expression in the KRAP-knockdown cells compared with the non-KRAP knockdown cells. Moreover, it has been made clear that the KRAP-knockout mice produced by the present inventors cause a remarkable systemic energetic metabolic disorder, and the KRAP protein possesses functions for raising energy consumption, accelerating sensitivity to insulin, preventing and controlling obesity, and/or inducing a disorder of blood hormone concentrations (Patent document No. 1 and Non-patent literature document No. 3). Therefore, agents targeting KRAP or KRAP-associated routes may provide the possibility for preventing and treating obesity or metabolic diseases including diabetes mellitus.

In order to explicate the molecular function of the KRAP protein, the present inventors have produced mice overexpressing KRAP and identify a protein purified from the liver tissue from the resulting mice, which interacts with the KRAP protein. That protein was identified as inositol 1,4,5-triphosphate receptor (IP3R)-binding protein, and the KRAP protein interacting with inositol 1,4,5-triphosphate (IP3) receptor has an important action for regulating IP3R.

### REFERENCES

### Patent Document

[Patent Document No. 1] WO 2007/010999

### Non-Patent Literature Documents

[Non-patent Literature Document No. 1] Inokuchi J, Komiya M, Baba I, Naito S, Sasazuki T, Shirasawa S., Deregulated exression of KRAP, a novel gene encoding actin-interacting protein, in human colon cancer cells. J Hum Genet. 2004; 49(1):46-52.
[Non-patent Literature Document No. 2] Fujimoto T, Koyanagi M, Baba I, Nakabayashi K, Kato N, Sasazuki T, Shirasawa S., Analysis of KRAP expression and localization, and genes regulated by KRAP in a human colon cancer cell line. J Hum Genet. 2007; 52(12):978-84.
[Non-patent Literature Document No. 3] Fujimoto T, Miyasaka K, Koyanagi M, Tsunoda T, Baba I, Doi K, Ohta M, Kato N, Sasazuki T, Shirasawa S. Altered energy homeostasis and resistance to diet-induced obesity in KRAP-deficient mice. PLoS One. 2009; 4(1): e4240.

### SUMMARY OF INVENTION

The instant invention has the object to provide an inositol 1,4,5-triphosphate receptor-binding protein, preferably inositol 1,4,5-triphosphate receptor (IP3R)-binding KRAP protein.

In a preferred embodiment, the instant invention has the object to provide an the IP3R-binding protein comprising an amino acid sequence constituting a protein conjugated with full length IP3R or a region carrying an IP3R binding site carrying an amino acid site to which IP3R is conjugated, that is, an IP3R binding site-carrying region.

In a more preferred embodiment, the instant invention has the object to provide the IP3R-binding protein in which a region containing IP3R binding site is located on an amino acid sequence region consisting of 19 amino acids starting with the amino acid 200 to the amino acid 218 of the mouse KRAP protein, or an amino acid sequence region corresponding to the an amino acid sequence region of the mouse KRAP protein in the case of the KRAP protein derived from an animal species other than mouse.

In a more preferred embodiment, the instant invention has the object to provide the IP3R-binding protein that is a structural mimic synthesized on the basis of structural information of an primary or higher amino acid structure of the IP3R-binding protein or the region carrying the IP3R conjugation site on which to be interacted with the protein.

In a more preferred embodiment, the instant invention has the object to provide an intracellular calcium ion modifier including, but being not limited to, an IP3R function modifier, an IP3R activation inhibitor or an IP3R activator, which contains the IP3R-binding protein or its IP3R binding site-carrying region as an active ingredient.

In a more preferred embodiment of the instant invention has the object to provide an assay for screening a substance inhibiting the protein-protein interaction between the KRAP-IP3R proteins, which comprises detecting a binding ability of a KRAP-IP3R protein complex composed of the KRAP protein and the IP3R protein or the IP3R binding site-carrying region thereof.

In a more preferred embodiment of the instant invention, the object is to provide a non-human transgenic mammalian animal overexpressing the IP3R-binding protein, KRAP, and furthermore a vector carrying the IP3R-binding protein.

The terms “inositol 1,4,5-triphosphate receptor-binding protein” or its related representations used in this description is or are intended to mean a KRAP-IP3R complex in which the IP3R protein is associated with the KRAP protein or analogues thereof, or a KRAP-IP3R complex in which nucleotide sequences or amino acid sequences structuring the IP3R protein are conjugated to the KRAP protein or analogues thereof, respectively.

The term “binding” of the terms “binding protein” and its related representations used herein is or are intended to mean a conjugation in the form of association or the like by the intermolecular action between nucleotide sequences or amino acid sequences, as well as proteins.

In order to achieve the above objects, the instant invention provides an inositol 1,4,5-triphosphate receptor-binding protein, preferably inositol 1,4,5-triphosphate receptor (IP3R)-binding KRAP protein.

In a preferred embodiment of the instant invention, there is provided the IP3R-binding protein comprising an amino acid sequence constituting a protein binding to full length IP3R or a region carrying an IP3R binding site composed of an amino acid site with which IP3R is associated. This region will also be referred to hereinafter as “IP3R binding site-carrying region”.

In a more preferred embodiment of the instant invention, there is also provided the IP3R-binding protein in which the IP3R binding site-carrying region is located on an amino acid sequence region consisting of 19 amino acids starting with the amino acid 200 to the amino acid 218 of the mouse KRAP protein, or an amino acid sequence region corresponding to the amino acid sequence region of the mouse KRAP protein in the case of the KRAP protein derived from an animal species other than mouse.

In a more preferred embodiment of the instant invention, there is further provided the IP3R-binding protein that is a structurally mimic compound synthesized on the basis of structural information of an primary amino acid structure or a higher amino acid structure of the IP3R-binding protein or the IP3R binding site-carrying region.

The instant invention in its another mode provides an intracellular calcium ion modifier including, but being not limited to, an IP3R function modifier, an IP3R activation inhibitor or an IP3R activator, which contains the IP3R-binding protein or its IP3R binding site-carrying region as an active ingredient.

The instant invention in its still another mode provides an assay for screening a substance inhibiting the interaction between the KRAP protein and the IP3R protein, which comprises detecting a binding ability of the KRAP-IP3R protein complex composed of the KRAP protein and the IP3R protein or the IP3R binding site-carrying region thereof.

In a still another mode, the instant invention provides a non-human transgenic mammalian animal overexpressing the IP3R-binding protein, KRAP, and furthermore a vector carrying the IP3R-binding protein.

To date, as agents capable of inhibiting IP3R activity, there are known heparin, xestospongin and 2-aminoethoxydiphenyl borate (2-APB). Heparin has a problem with the specificity against IP3R inhibition because it is also known to uncouple G protein signaling or activate ryanodine receptor. It is also required to be injected into cells because it possesses no permeability to membrane. Xestospongin is a natural alkaloid derived from a natural sponge so that it has drawbacks due to its expensive costs for preparation. The mechanism how it inhibits the Ca²⁺ signaling is not fully clarified. 2-APB has also problems with specificity because it possesses the property of suppressing Ca²⁺ release from the endoplasmic reticulum storage and acting on the store-operated Ca²⁺ channel on the plasma membrane. On the other hand, the IP3R-binding protein according to the instant invention possesses the action and effects for modifying the functions of IP3R due to its intermolecular action of the proteins such as the KRAP protein on the IP3R protein. Therefore, the IP3R-binding protein can be expected to become an IP3R activity modifier of a new type that is different from the action and effects possessed by conventional IP3R activity modifiers.

Moreover, the instant invention can be utilized for the development of a new IP3R function modifier as well as a new intracellular calcium ion concentration modifier using the substance mimicking the primary amino acid structure or a higher steric structure of the amino acid sequence or its IP3R binding site-carrying region of the protein such as KRAP protein.

### BRIEF DESCRIPTION OF DRAWINGS

FIG. 1 shows the results of PCR using ex Taq enzyme, in which the genomic DNAs extracted from the tails of a wild type mouse (WT) and an established transgenic mouse (TG) were used as a template.

FIG. 2 shows the results obtained by Western blotting of the protein solution prepared from each of the tissues of the KRAP transgenic mouse and detection with anti-HA antibody.

FIG. 3 shows a relative KRAP expression level of the liver of the transgenic mouse compared with that of a wild type one.

FIG. 4 illustrates immunohistochemical staining analysis of the localization of the overexpressed KRAP protein in the mouse liver using anti-HA antibody and Phalloidin.

FIG. 5 illustrates silver staining analysis of HA-matrix binding fractions developed by SDS-PAGE, which were prepared from the livers of a wild type (WT) mouse and transgenic (TG) mouse, respectively.

FIG. 6 shows analysis of bands of mouse inositol 1,4,5-triphosphate receptor 1 using a mass spectrometer.

FIG. 7 shows Western blot analysis of the co-purified products.

FIG. 8 shows Western blot analysis of immune precipitation products from the protein extracts of the tissues (liver, kidney, brown adipose tissue (BAT), and pancreas) using anti-KRAP antibody.

FIG. 9 shows Western blot analysis of immune precipitation products from the protein extracts of cultured cells (HCT116 and Hela cells) using anti-KRAP antibody.

FIG. 10 shows immunostaining analysis of cultured cells (HEK 293 cells) for observation of the co-localization of the proteins.

FIG. 11 shows representative illustrations of periodical variations in calcium indicator-fluorescence signals in cultured cells (HEK293) pretreated with KRAP-specific siRNA or control-siRNA. The signals were recorded before and after the stimulation of cells with ATP concentrations indicated. In each figure, the upper line shows the cells treated with scramble siRNA and the lower line shows the cells suppressing the KRAP expression.

FIG. 12 shows a comparison of maximum fluorescence values after ATP stimulation at the final ATP concentrations of 1 μM, 3 μM and 10 μM, respectively. In each figure, the left-hand bar shows the cells treated with scramble siRNA and the right-hand bar shows the cells suppressing the KRAP expression.

FIG. 13 shows mean peak amplitudes of the maximum fluorescence values of the Ca²⁺ response as expressed by a ratio when the maximum fluorescence value before the ATP stimulation is represented as 1. The left-hand bars show the cells treated with scramble siRNA, and the right-hand bars show the cells suppressing the KRAP expression.

FIG. 14 shows total fluorescence values after stimulation expressed by addition of fluorescence values measured during a period of from 3 seconds to 25.5 seconds after ATP stimulation. The left-hand bars show the cells treated with scramble siRNA, and the right-hand bars show the cells suppressing the KRAP expression.

FIG. 15 illustrates Western blot analysis of the suppression of the KRAP expression in HEK 293 cells.

FIG. 16 is schematic diagrams of the mouse KRAP protein. The diagrams from top to bottom show full length, deletion mutant KRAP construct deficient solely in a coiled coil (988-1252), and deletion mutant KRAP constructs with the N-terminal region shortened little by little (1-318, 1-236, 1-218, and 1-198). To each of the N-terminus and the C-terminus is added an HA tag.

FIG. 17 illustrates analysis of electropherograms of the binding capacity of the KRAP full length and each deletion mutant to IP3R in HEK 293 cells.

FIG. 18 illustrates analysis of electropherograms of the binding capacity of the KRAP full length and each deletion mutant to IP3R in HCT 116 cells.

FIG. 19a shows amino acid sequences of mouse KRAP and human KRAP in a region of 19 amino acids which is important as a KRAP protein region for binding to IP3R, which was identified in Example 5 below. FIG. 19b illustrates analysis of electrophorogram of the binding capacity of a spot mutant to IP3R1.

### MODES FOR CARRYING OUT THE INVENTION

The instant invention relates to an inositol 1,4,5-triphosphate receptor (IP3R) binding protein, an intracellular calcium ion concentration modifier including, but being not limited to, an IP3R function modifier, an IP3R activity inhibitor, an IP3R activator or the like, and a non-human transgenic mammalian animal containing the IP3R protein.

The IP3R-conjugated protein according to the instant invention is located widely in a variety of tissues and cells in the form of an intracellular conjugation between the IP3R protein and the protein including, but being not limited to, the KRAP protein.

A description will be made regarding inositol 1,4,5-triphosphate (IP3) and the IP3 receptor (IP3R) with IP3 conjugated thereto as a ligand. IP3 is a signaling molecule to be produced upon response to a number of extracellular stimulation such as hormones, growth factors, neurotransmitters and so on, and it is a second messenger controlling the cellular functions dependent upon a variety of calcium ions by induction of the calcium ion release from the organelle (mainly endoplasmic reticulum) acting as a calcium ion storage in the cell. IP3R plays an important role in the control of a variety of cell functions including, for instance, secretion, fertilization, muscle contraction, nerve signaling, cell growth and so on (Berridge, M., et al., Nature, 361, 315, 1993; Claphem, D. E., Cell, 80, 259, 1995).

Further, IP3 possesses the action for increasing the calcium ion concentration in the cell by activation of IP3R present on the membrane of the endoplasmic reticulum or the like acting as a calcium ion storage upon conjugation to IP3R as a ligand, and by induction of release of the calcium ion. It is to be noted herein that the calcium ion acts an important function as an intracellular transmitter, and the calcium ion concentration in the cell is kept normally at a level as very low as approximately one-thousandth compared to that outside the cell. A variation in the calcium ion concentration is involved in various cell responses including cell division, apoptosis, fertilization, development, and so on.

On the other hand, IP3R receptor (IP3R) is widely distributed in a variety of tissues and cells including the brain, heart, liver, kidney, pancreas, thymus gland and so on of mammalian animals such as human, mouse, etc. It is also present widely in excitatory cells including the nerve, muscle, etc. and non-excitatory cells, and it is further involved in diverse biological phenomena. The IP3 receptor is an intracellular IP3-gated Ca²⁺ release channel in the form of a tetramer and consists of three functionally different domains, i.e., an IP3 binding domain located in the vicinity of the N-terminus, a channel formation domain with a six-transmembrane domain located in the vicinity of the C-terminus, and a regulatory domain located between the two domains. IP3R has so far been extensively studied in terms of its localization in the brain. There are three distinct isoforms, i.e., type 1, 2, and 3, each of which is localized in the brain. Among them, in particular, IP3R type 1 (IP3R1) is present abundantly in the Purkinje's cells of the cerebellum.

The IP3R-binding protein is one of binding proteins in which a protein such as KRAP protein or the like is associated with the IP3R protein by intermediation of an intermolecular action. For instance, IP3R-binding KRAP protein can be obtained by co-purification of various tissues (for example, liver tissue, etc.) of transgenic non-human mammals such as a KRAP protein-overexpressing transgenic mouse produced by the present inventors.

In the instant invention, the nucleotide sequence of human KRAP protein is indicated as SEQ ID NO. 1; the human amino acid sequence of the human KRAP protein as SEQ ID NO. 2; the nucleotide sequence of mouse KRAP protein as SEQ ID NO. 3; the amino acid sequence of the mouse KRAP protein as SEQ ID NO. 4; the nucleotide sequence of human IP3R protein as SEQ ID NO. 5; the amino acid sequence of the human IP3R protein as SEQ ID NO. 2; the nucleotide sequence of mouse IP3R protein as SEQ ID NO. 7; and the amino acid sequence of the mouse IP3R protein as SEQ ID NO. 8.

For instance, the mouse KRAP protein (SEQ ID NO. 3) to be associated with IP3R is composed of 1,252 amino acids and presumed in such a fashion that its C-terminal region has a coiled-coil structure. The IP3R-binding KRAP protein according to the instant invention is to be understood that not only its full length nucleotide/amino acid sequence of the KRAP protein but also its nucleotide /amino acid sequences shorter than the full length thereof fall within the scope of the invention, which carries a site with which the IP3R protein is associated, i.e., its IP3R-binding KRAP protein-carrying regions, as far as they can demonstrate an activity substantially equal to that of the full length. The region of the KRAP protein significant for binding to IP3R can be determined by investigation of the binding capacity of a KRAP deletion mutant to the IP3R protein. The investigation of the binding capacity of the KRAP deletion mutant to the IP3R protein may be conducted, for instance, by expressing the full-length KRAP protein or the KRAP deletion mutant together with IP3R in HEK 293 cells, subjecting the protein extract from the resulting HEK 293 cells to immunoprecipitation with anti-HA antibody, and determining an occurrence of the precipitation of the KRAP protein together with IP3R. The IP3R binding site-carrying region identified in the above manner as being considered important for binding to IP3R is considered as composed of 191 amino acids located at from amino acid 200 to 218 of the mouse KRAP protein. Therefore, an amino acid sequence region of the KRAP protein encompassing this region is also to be understood to fall within the scope of the invention.

As described above, the region composed of the above 19 amino acids of the mouse KRAP protein is considered important for binding to the IP3R protein. The region of the human KRAP protein corresponding to the mouse KRAP protein is considered accordingly to be important for binding thereto.

In accordance with the instant invention, the structural mimics and equivalent compounds to be formed on the basis of information on the primary or higher amino acid conformations of the IP3R-KRAP protein complex comprising the IP3R-binding KRAP protein or its IP3R binding site-carrying regions are also to be understood to fall within the scope of the invention, as a matter of course, because they possess and demonstrate substantially the same or equivalent functions and actions as the IP3R-binding KRAP protein or its IP3R binding site-carrying regions can.

The substances containing the IP3R-binding KRAP protein or its IP3R binding site-carrying amino acid regions may be utilized for development of an intracellular calcium ion concentration modifier including, but being not limited to, an IP3R function modifier, an IP3R inhibitor, an IP3R activator, or the like.

Moreover, the IP3R-KRAP protein complex with the KRAP protein binding to the IP3R protein may be utilized for an assay for screening substances for inhibiting the interaction between the IP3R and KRAP proteins by detecting the binding capacity of the IP3R-KRAP protein complex.

As the transgenic non-human mammalian animal according to the instant invention, there may be mentioned, for example, monkeys, cattle, swine, dogs, cats, rabbits, Guinea pigs, rats, hamsters, mice, and the like, although all mammalian animals may be included in a technical point of view. Rodents such as Guinea pigs, rats, hamsters, mice, and the like are preferred from a viewpoint of producing, breeding, and brevity of use. In particular, mice are most preferred because a large number of inbred strains have already been produced, and technology has already established well with respect to cultures of fertilized eggs, in vitro fertilization, and so on.

The following is a description regarding a method for the production of the transgenic non-human mammalian animal according to the instant invention. For a brevity of explanation, the description will be made by taking a mouse as an example of the non-human mammalian animal. It is however to be noted herein as a matter of course that the instant invention is not interpreted to be limited to the mouse, and, unless otherwise stated, it is intended to encompass the other non-human mammalian animals.

The transgenic non-human mammalian animals according to the instant invention may be produced, for example, by introducing the gene of interest into the transgenic non-human mammalian animals in accordance with techniques as used conventionally in the art (for example, Proc. Natl. Acad. Sci. USA 77; 7380-7384, 1980).

For the production of the transgenic non-human mammalian animals according to the instant invention, first, the gene to be introduced therein is formed from cDNA of the KRAP gene. The KRAP cDNA can in turn be prepared, for instance, by a method involved in synthesizing an oligonucleotide on the basis of a nucleotide sequence part of a known mouse cDNA sequence or a human cDNA sequence and screening a cDNA library using the resulting oligonucleotide as a probe, or in isolating mRNA from normal cells or cultured cells, etc., for synthesizing oligonucleotides as a forward primer and a reverse primer for hybridization with the both termini of the cDNA fragment of interest, and preparing the KRAP cDNA by RT-PCR using these primers. To the 5′-terminal and 3′-terminal sites, there may be added each an appropriate restriction enzyme sequence so as to template with an insertion site of an expression vector as will be described later. To the gene to be introduced, a promoter or an enhancer may also be connected. The promoter and the enhancer may not be limited to a particular one, and there may be appropriately selected and applied a promoter sequence and an enhancer sequence of a gene highly expressed in various organs of the animal into which the KRAP cDNA is to be introduced.

It is generally well known in the art that the amino acid sequence of a protein possessing a physiological activity may keep its physiological activity even if one or a very small number of amino acids would be substituted, deleted, inserted or added. Therefore, a gene coding for a polypeptide carrying an amino acid sequence with one or a very small number of amino acids of the known KRAP amino acid sequence substituted, deleted, inserted or added and possessing an original KRAP activity, may also be used in substantially the same manner as normal one.

In order to enhance the expression efficiency, the gene obtained by cloning the gene to be introduced may be led to an expression construct by introduction into an expression vector carrying an appropriate promoter. As the gene to be introduced, there may be preferably used a recombinant gene connected downstream of an appropriate promoter as conventionally used for mammals, and furthermore a polyA signal may also be preferably connected downstream of the gene involved. To the recombinant gene may be connected a terminator necessary for the expression of the gene encoding the above peptide, and it may also be used as a sequence (i.e., a so-called polyA) for terminating the transcription of the mRNA of interest.

The promoter and the polyA signal are not limited to particular ones, respectively. The promoters may include, but be not limited to, virus-derived promoters from cytomegalovirus, JC virus, breast cancer virus and the like as well as mammal-derived promoters from humans, rabbits, dogs, cats, Guinea pigs, hamsters, rats, mice and the like. Among the promoters, for example, there may be preferably used, pCAGGS vector, i.e., a so-called CAG promoter, carrying the structure with a cytomegalovirus enhancer connected to a chicken-β-actin promoter, and a polyA signal site of rabbit β-globin gene, because it may nearly systemically overexpress the gene of interest. As the terminator, there may be preferably used, for example, SV40 terminator of simian virus, or the like.

The expression construct prepared in the above manner may be injected into Escherichia coli and amplified by incubating E. coli. After purification, the nucleotide sequence may be preferably confirmed with a sequencer.

In the instant invention, the expression construct may be generally injected by microinjection into a pronucleus which is formed before fusion of the nuclei of the ovum and sperm of a fertilized egg of a non-human mammal, and the gene of interest is introduced into the pronucleus. Upon introduction of the gene of interest, a plasmid (construct) may be introduced in a ring form or a linearized form, however, there may be generally preferred such a linearized form in which a structuring gene region and an expression regulation region such as a promoter, etc., are not broken. The fertilized egg with the gene of interest introduced therein is then transplanted to pseudopregnant male mice, and baby mice are delivered to produce transgenic mice.

As a method for the introduction of the gene of interest into the fertilized mouse eggs other than the above method, there may be used, for example, the method of introduction into ES cells and the method of introduction of a cell nucleus into cultured cells and then transplantation to fertilized egg. In the above methods, a vector with the genomic DNA of interest incorporated therein may then be introduced into the ES cells and the cultured cells, respectively, by conventional techniques such as electroporation or lipofection, and positively selected with neomycin, puromycin or the like, to produce the introduced cells of interest. The ES cells may be injected into blastocyst embryos or eight-cell stage embryos of mice by microinjection or the like. The transplantation of the nucleus may be carried out by injecting the cell carrying the genomic DNA of interest into the fertilized egg with the nucleus excluded therefrom and conducting cell fusion by electrical stimulation.

The blastocyst embryos or eight-cell stage embryos with the ES cells injected therein may be then transplanted directly to the oviduct of a foster mother mouse or the womb of a foster mother mouse after generation of a blastocyst by incubation for one day. Breeding of the foster mother mice and giving birth to newborn mice may produce transgenic mice (chimeric animals) with the gene of interest introduced therein. Whether the newborn mice carry the gene of interest may be confirmed by subjecting the genomic DNA extracted from somatic cells of a tissue part (e.g., tail tip) to PCR or Southern blotting. For instance, the existence of the gene of interest in the newborn mice may be confirmed by using the genomic DNA prepared from the mouse tail as a template and subjecting it to PCR with ex Taq enzyme using appropriate primers.

The introduced gene of interest held by the newborn mouse is present in all cells in equal amounts, and whether it is expressed in the objective tissue may be confirmed by investigating the expression as RNA by RT-PCR. Moreover, whether it is produced as a protein may also be confirmed by subjecting the resulting protein or its partial peptide to immunoblotting with an antibody thereto.

The individual transgenic mouse (founder) in which the overexpression of the introduced gene of interest is confirmed may be then crossed with a normal animal, thereby producing a heterozygotic animal (F1). The heterozygotic animals (F1) are further crossed with each other to produce homozygotic animals (F2). The long-term passage by crossbreeding the homozygotic animals of the identical species in a normal breeding environment may result in maintenance of the gene of interest in the germ line in a stable fashion.

The IP3R-binding protein according to the instant invention may be identified by co-purifying the tissue of the organs, e.g., liver of a mouse, in which the gene of interest prepared in the manner as above is overexpressed. The method for co-purification may include, but be not limited to, immunoprecipitation method using an antibody to the protein of interest. This may enable confirmation of association of the endogenous KRAP protein with the endogenous IP3R protein of the protein extract from the living mouse tissue or cell line.

The following is a further detailed description regarding the IP3R-binding protein and the transgenic non-human mammalian animal according to the instant invention, carrying the IP3R-binding protein, by way of working examples. It has to be understood herein that the instant invention is not at all interpreted to be limited to the following working examples, and the working examples below will be described solely for the purpose of illustration of the invention. It is also to be understood that every and all variations and modifications may be encompassed within the scope of the instant invention, which can be readily inferred from this description and the following working examples.

### EXAMPLE 1

A full length sequence of mouse KRAP coding region was inserted into Xhol-Bgl II portion of pCAGGS vector (Gene 1991, Niwa, H., et al.), whereby forming an expression construct. The insert was prepared by adding the Xhol recognition sequence and the 5′-sequence 10b of the mouse KRAP gene to the 5′-portion of the coding region thereof as well as the HA tag, stop codon and BamHI recognition sequence to the 3′-portion thereof. The insert was prepared by PCR using mouse KRAP cDNA registered as accession No. AB120565 as a template and LA-taq enzyme (Takara). The forward primer and the reverse primer used herein are as follows:

Forward primer (SQ ID NO. 9):
5′-gggctcgagcggcgcggccatgaaccgacccctgtcg-3′
Reverse primer (SQ ID NO. 10):
5′-gggggatcctcaagcgtaatctggaacatcgtatgggtagtggctg
tcctgcttaggacc-3′

### EXAMPLE 2

A full length sequence of mouse KRAP coding region with HA tag added to its C-terminus was cloned on pcDNA/V5/GW/D-TOPO vector (Invitrogen), thereby forming an expression construct. The insert was prepared by PCR using mouse KRAP cDNA registered as accession No. AB120565 as a template and LA-taq enzyme (Takara). The forward and reverse primers used herein are as follows:

Forward primer (SQ ID NO. 11):
5′-caccatgaaccgacccctgtcg-3′
Reverse primer (SQ ID NO. 12):
5′-ctactaagatctctaagcgtagtctgggacgtcgtatgggtagtgg
ctgtcctgcttagg-3′

### EXAMPLE 3

The deletion mutants were prepared by subjecting DNA fragments coding for regions of amino acid residues 988-1252 (coiled-coil region only), amino acid residues 1-318, amino acid residues 1-236, amino acid residues 1-218, and amino acid residues 1-199, respectively, to PCR and cloning them to cloning site SalI-NotI of pCMV-HA vectors (Clonetech). They were constructed in such a form that a stop codon was inserted in the C-terminus of the KRAP fragment by linking the N-terminal HA tag carried in the vector to the open reading frame.

### EXAMPLE 4

The point-mutants were prepared using KOD-Plus-Mutagenesis Kit (TOYOBO) on the basis of the full length construct of the KRAP coding region prepared above on pcDNA/V5/GW/D-TOPO vector: F202A and F203A mutants with phenylalanine at the amino acids 202 and 203 counted from the amino acid 1 replaced, respectively, by alanine, as well as F202A/F203A mutant with the above two residues replaced by alanine. The primers used herein are as follows:

Forward primer (for F202A) (SQ ID NO. 13):
5′-gcatttaattcatcatcctttgccagaggc-3′
Reverse primer (for F202A) (SQ ID NO. 14):
5′-tctggaaggaattttagaagcaatatctg-3′
Forward primer (for F203A) (SQ ID NO. 15):
5′-gcaaattcatcatcctttgccagaggc-3′
Reverse primer (for F203A) (SQ ID NO. 16):
5′-aaatctggaaggaattttagaagcaat-3′
Forward primer (for F202A/F203) (SQ ID NO. 17):
5′-gcagcaaattcatcatcctttgccagaggc-3′
Reverse primer (for F202A/F203) (SQ ID NO. 18)
5′-tctggaaggaattttagaagcaatatctg-3′

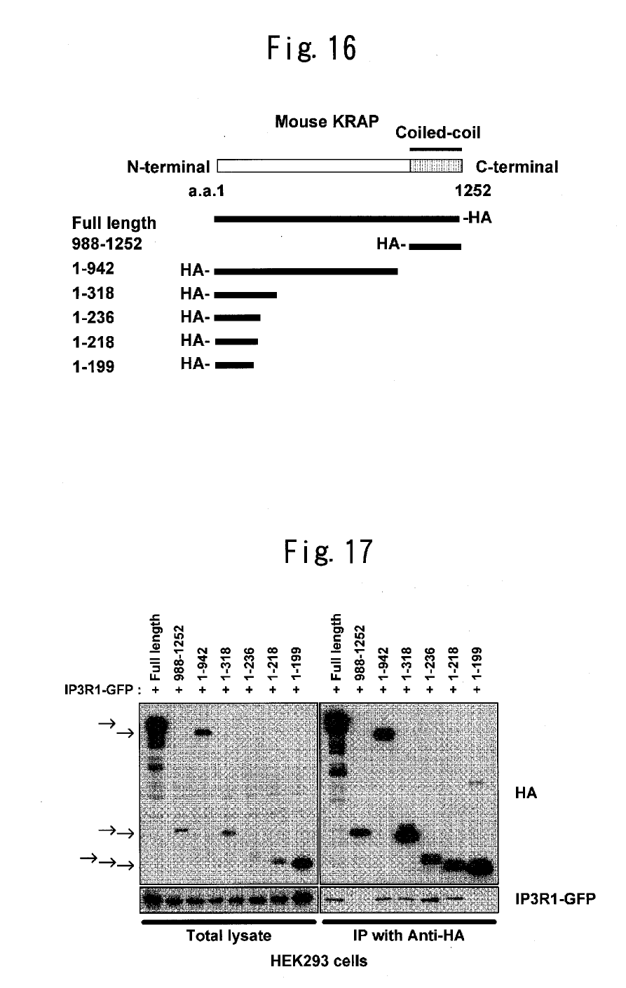

FIG. 16 is schematic diagrams showing the mouse KRAP protein. From top to bottom, the figures show the full length sequence; the deletion mutant construct composed of the coiled-coil region (988-1252) only; the deletion mutants with the coiled-coil region knocked out (1-942); as well as the deletion mutant constructs with the N-terminal region shortened (21-318, 1-236, 1-218, and 1-199). To each N-terminus or C-terminus was added HA tag.

### EXAMPLE 5

This example is an experiment conducted for the purpose to investigate the protein-protein interaction activity for determination of the KRAP protein region significant for binding to the IP3R protein. In this experiment, the binding capacity of each of the full length sequence and the deletion mutants to the IP3R protein was studied. The experiment was involved in expressing each of the full length sequence and the deletion mutants together with IP3R in HEK 923 cells and subjecting each of the protein extracts therefrom together with IP3R to immunoprecipitation using anti-HA antibody in order to determine the immunoprecipitation of IP3R.

Into HEK 293 cells, a full length IP3R1-GFP expression plasmid and each of the KRAP full length, the deletion mutants, or the point-mutant expression plasmids were introduced together with lipofectamine LTX (Invitrogen), respectively, and the resulting cells were incubated for 24 hours. They were then homogenized in an extracting buffer (50 mM Tris-HCl, pH7.5, 140 mM NaCl, 0.5% NP40, 0.25M NDSB-201 (Calbiochem), and protease inhibitors (Complete EDTA-free, Roche)), and the homogenized mixture was centrifuged at 15,000 rpm for 30 minutes. The resulting supernatant was then subjected to immunoprecipitation with HA matrix (Roche) to precipitate the IP3R-GFP protein binding to the KRAP protein, and the precipitated IP3R-GFP protein was detected by Western blots using a rabbit polyclonal antibody (Clonetech) as an anti-GFP antibody. In the experiment using HCT 116 cells, the presence or absence of co-precipitation of the endogenous IP3R protein was investigated without transfection of the IP3R-GFP protein using a mouse monoclonal antibody (BD Transduction Laboratory) as an anti-IP3R3 antibody.

FIG. 17 shows the results of the binding capacity of the KRAP full length and each of the deletion mutants to IP3R1 in HEK 293 cells. As shown in the figure, it was found that a nearly equal amount of the protein was recovered in the immune precipitated (IP) fractions from each of the deletion mutants using HA matrix. As a result of investigation of the co-precipitated IP3R1 protein, it was found that each of the KRAP full length and the amino acid regions (1-942) was precipitated with IP3R1 while the amino acid region (988-1252) corresponding to the coiled-coil region was not precipitated therewith. This revealed that the binding region existed in the N-terminal KRAP portion. As a result of further investigations on the mutants with a number of the N-terminal amino acids shortened little by little, it was found that the amino acid region (1-218) demonstrated the bind capacity, while the amino acid region (1-199) demonstrated no binding capacity. This result implies that the region composed of 19 amino acids located at the amino acid 200 to 218 is significant for conjugation to IP3R1.

### EXAMPLE 6

As it is known that IP3R is composed of a family of different genes, i.e., type 1, 2 and 3, and these types are expressed in different cells and tissues, this example was carried out to investigate as to whether the KRAP protein possesses the binding capacity to IP3R other than type 1, and the binding mode of the other types is in common.

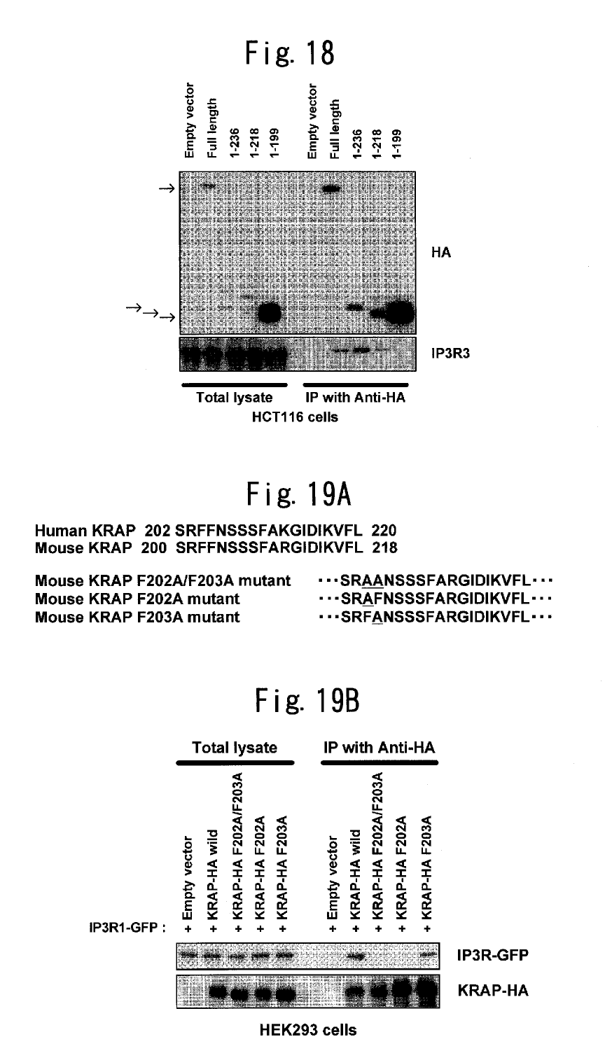

This experiment was carried out by expressing each of the deletion mutants in HCT 116 cells and subjecting it to immunoprecipitation with endogenous IP3R type 3 in substantially the same manner as in Example 5. As a result, it was found that the N-terminal amino acid region (1-218) of KRAP demonstrated the binding capacity to IP3R type 3, while the N-terminal amino acid region (1-199) demonstrated no binding capacity thereto. This makes it clear that the KRAP amino acid region may interact with the other types of IP3R in substantially the same fashion as the type 1 protein. FIG. 18 shows the results of the binding capacity of each of the KRAP full length and the deletion mutants to IP3R1 in HCT 116 cells.

### EXAMPLE 7

This example was carried out to investigate the binding of the KRAP protein region composed of 19 amino acids which was identified in Example 5 above as being significant for binding to IP3R.

FIG. 19a shows analysis of the amino acid sequences of the human KRAP amino acid sequence 202-220 (SQ ID NO. 19) and the mouse KRAP amino acid sequence 200-218 (SQ ID NO. 20) corresponding thereto, respectively. As shown therein, a comparison of these two sequences reveals that the human and mouse amino acid sequences are well conserved between the two species. The mutation sites of the point-mutants are underlined on the amino acid sequences of the mouse and human KRAP proteins, respectively. The point-mutants are shown in which phenylalanine located at the amino acids 202 and 203 counted from the N-terminus is substituted both with alanine (A) (mouse KRAP F202A/F203A mutant: SQ ID NO. 21) or either is substituted therewith (mouse KRAP F202A mutant: SQ ID NO. 22 and mouse KRAP F203A mutant: SQ ID NO. 23). In the figure, the underlined letter A (Ala) indicates a portion corresponding to F (Phe) of the wild type.

In order to determine the KRAP amino acid significant for binding to IP3R, the experiment regarding the binding capacity using the point-mutants was carried out in substantially the same manner as in Example 5. In this experiment, each of the wild type and the point-mutants was expressed together with IP3R1 in HEK 293 cells, followed by immunoprecipitation of the resulting protein extract with HA matrix to investigate whether IP3R1 was precipitated together therewith. As a result, it was found that the binding capacity of the spot mutant with phenylalanine (Phe) at the position of amino acid 202 substituted with alanine (Ala) was lost to a remarkable extent.

### EXAMPLE 8

In this example, a 6.2 kbp DNA fragment obtained by digestion of a plasmid with BamHI was injected into a fertilized egg by microinjection. The resulting fertilized egg was transplanted to the womb of a pseudopregnant female mouse, followed by giving birth to baby mice. Whether the introduced gene was kept in the baby mice was confirmed by extracting the genomic DNA from the tail and subjecting the DNA as a template to PCR using the following primers and ex Taq enzyme (Takara).

Forward primer (SQ ID NO. 24):
5′-gtcacagagctaatgcggga-3′
Reverse primer (SQ ID NO. 25):
5′-cttcacactgcagttgagtc-3′
and
Reverse primer (SQ ID NO. 26):
5′-ggcttcatgatgtccccat-3′

After confirmation as to the preservation of the gene of interest in the newborn baby mice, they were then crossed with each other to establish the germ line as C57BL/6J Jcl mice.

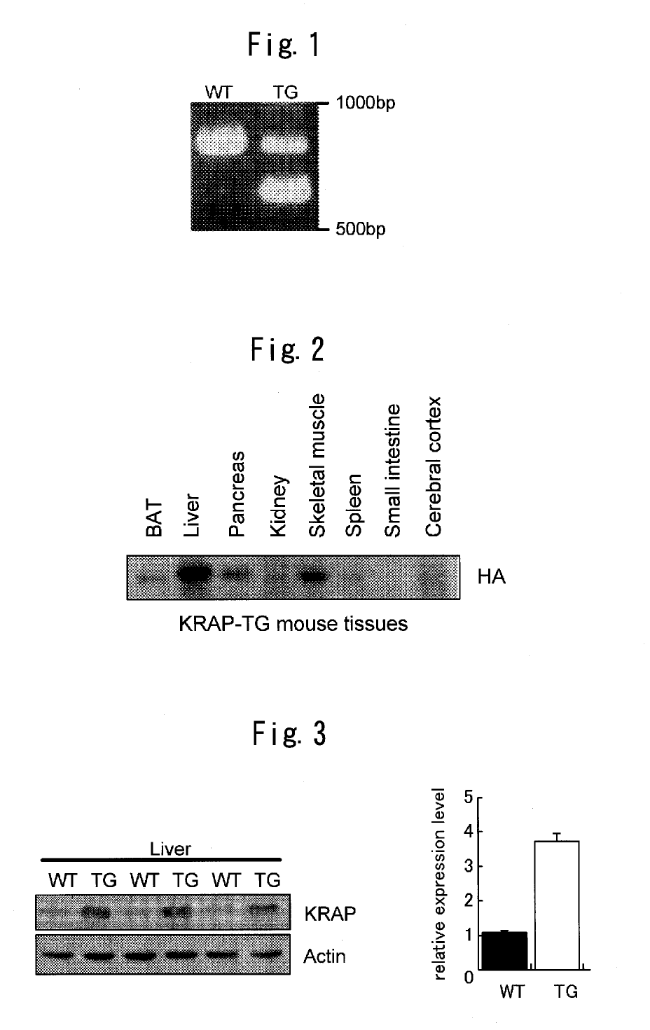

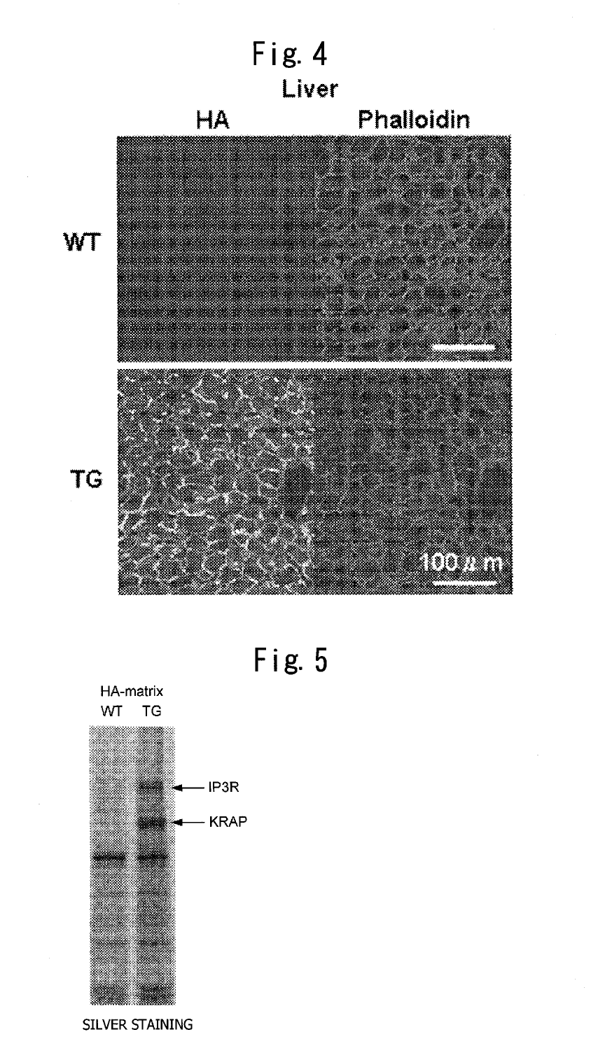

FIG. 1 shows the analysis of PCR using the genomic DNA obtained from the tail of the established transgenic mouse as a template, ex Taq enzyme, and the following primers. FIG. 1 reveals that the PCR product was amplified to the fragment having the size as anticipated, i.e., to 892 by for the wild type (WT) and 636 by for the transgenic mouse (TG).

Each tissue or cells of the resulting transgenic mouse was homogenized in an extracting buffer (50 mM Tris-HCl, pH 7.5, 150 mM NaCl, 1% NP40, 0.5% deoxycholate, 0.1% SDS, and protease inhibitor (Complete EDTA-free, Roche)), and centrifuged at 15,000 rpm for 30 minutes, followed by recovering the supernatant and yielding a total lysate. The resulting protein solution was then subjected to Western blot in a conventional manner to yield a protein: i.e., dissolving the resulting protein by SDS-PAGE, transferring it on a nitrocellulose membrane or PVDF membrane, and subjecting the membrane to immunoblotting with an anti-HA antibody to detect the protein (FIG. 2). In the Western blot, anti-HA antibody (3F10 clone; Roche), anti-KRAP (J. Hum. Genet. 2007, Fujimoto T. et al.), Anti-IP3R1 (Sigma), Anti-phospho-IP3R (Ser1756; Cell Signaling), Anti-phospholipase Cy (Santacruz), and Anti-actin (Sigma) were used primary antibodies. A HRP labeled antibody was used a secondary antibody. The detection method was based on chemiluminescence using an ECL reagent (GE Health Science).

FIG. 2 shows the analysis of detection of the protein mixture prepared from each tissue of the transgenic mouse by Wester blot using anti-HA antibody. The results as shown in FIG. 2 reveal that a relatively strong expression of the KRAP-HA protein was observed in the liver, pancreas, skeletal muscle, and brown adipose tissue, while a weak expression was observed in the kidney, spleen, and cerebral cortex. In the liver, an approximately four-fold increase in KRAP overexpression was confirmed in the TG mouse compared with that in the wild type mouse.

The localization of the overexpressed KRAP protein in the mouse liver was investigated by immunohistochemical staining with anti-HA antibody. The mouse liver was frozen in OCT compound (Sakura) and cut into frozen sections with a cryostat. The section was fixed with 4% PFA and blocked with a blocking solution (5% bovine serum, 20 mM Tris-HCl (pH 7.5), 150 mM NaCl, and 0.1% Tx-100), followed by staining with anti-HA (3F10 clone, Roche). An Alexa Flour 488 labelled anti-rat antibody (Invitrogen) was used as a secondary antibody, and Phalloidin-Alexa Fluor 546 (Invitrogen) was used for F-actin staining.

The immunohistochemical staining of the overexpressed KRAP protein in the liver with anti-HA antibody revealed that the KRAP protein was localized in the liver in the vicinity of the apical membrane, the basement and the sides of the parenchymal cell. This localization was found coincident with the localization of the endogenous KRAP previously reported by the present inventors (J. Hum. Genethe IP3R conjugation site 2007, Fujimoto T. et al.), and the overexpressed protein is considered as existing in the originally functional part.

The KRAP protein was identified by purification of the liver extract of the KRAP transgenic mouse (TG). The liver extract was collected from the liver of the wild type mouse (WT) and the KRAP transgenic mouse (T) and homogenized in an extracting buffer (50 mM Tris-HCl (pH 7.5), 140 mM NaCl, 0.5% NP40, 0.25 M NDSB-201 (Calbiochem), and protease inhibitor (Complete EDTA-free, Roche)), followed by centrifuging the resulting homogenized mixture at 18,500 rpm for 30 minutes and yielding a supernatant. To the resulting supernatant was added anti-HA affinity matrix (Roche), and the mixture was mixed at 4° C. for 5 hours. After the affinity matrix was washed with the extracting buffer and further with PBS, the mixture was boiled with SDS-PAGE sample buffer to yield an eluate franction. The resulting eluate fraction was then dissolved by SDS-PAGE and silver stained with SilverQuest (Invitrogen). The band precipitated with the KRAP-HA protein was cut, and the protein was identified by mass spectrometry. FIG. 5 shows silver-stained patterns obtained by developing the HA-matrix conjugated fraction prepared from the liver of the wild type mouse (WT) and the KRAP transgenic mouse (TG) by SDS-PAGE. As shown in FIG. 5, the band of the KRAP protein with HA tag added thereto and the band purified together with KRAP were detected.

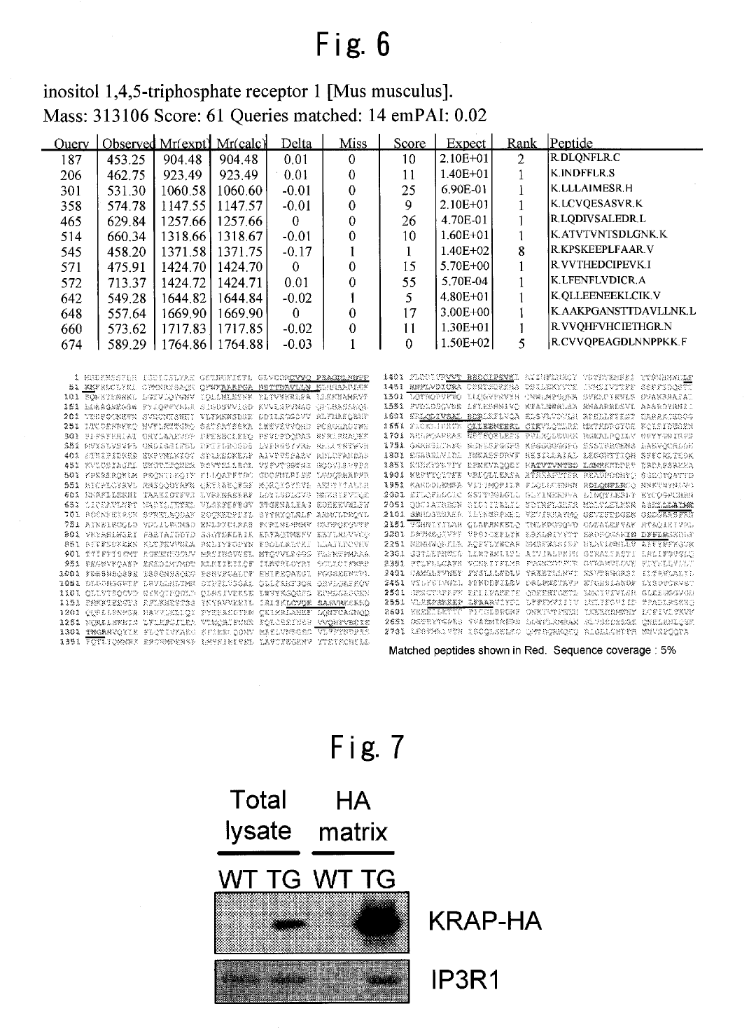

The analysis of the protein bands resulting from co-purification by mass spectrometry reveals that they coincide with the mass of the digested material of inositol 1,4,5-triphosphate 1 (IP3R1) at 13 positions (FIG. 6).

The Western blots of the co-purified product confirmed that the band was IP3R1 because it was detected with the anti-IP3R antibody.

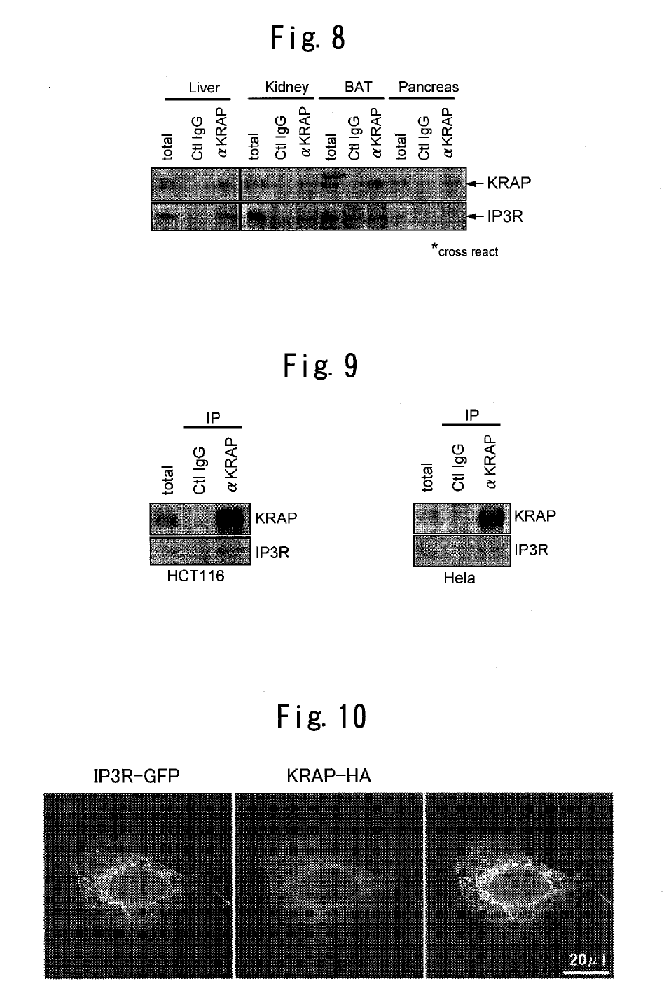

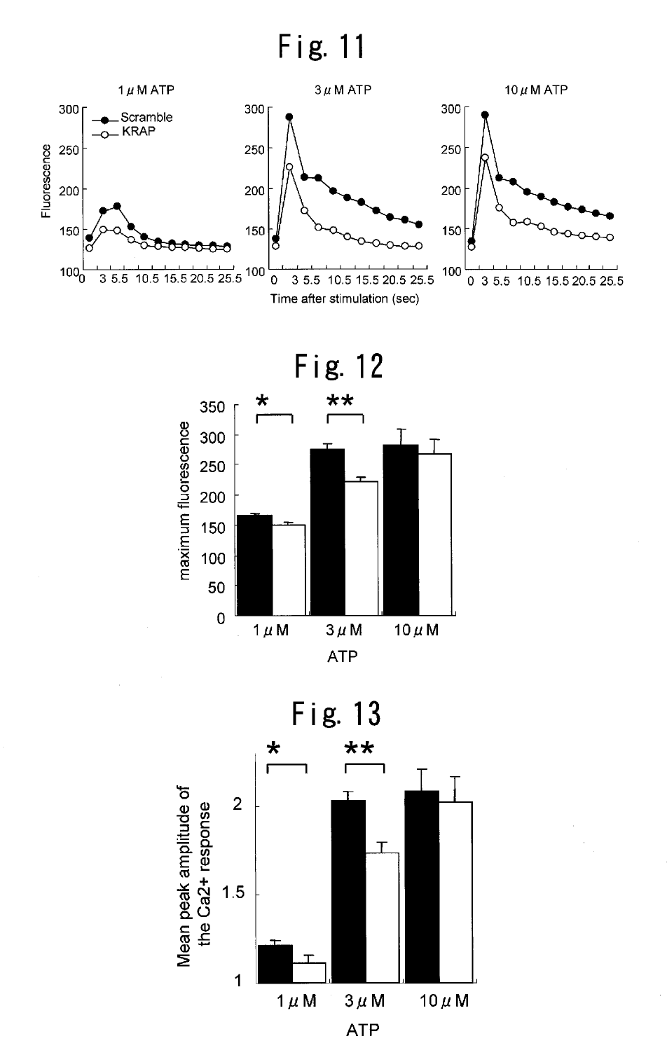

In order to ensure the presence or absence of the physical interaction between the endogenous KRAP protein and the endogenous IP3R1 protein, the precipitated materials obtained by immunoprecipitation of the protein extract from the tissue (liver, kidney, brown adipose tissue (BAT), pancreas) and cultured cells (HCT 116 cells and Hela cells) with anti-KRAP antibody were investigated by Western blots as to whether the IP3R1 protein was contained in the precipitated materials. The Western blots revealed that significant bands of IP3R1 protein were detected in both bands (FIGS. 8 and 9). As no band of the IP3R1 protein was detected in the material immunoprecipitated using control IgG, it is considered that the IP3R1 protein was precipitated together with the KRAP protein to be associated thereto.

The mouse tissue or the cultured cells were homogenized in an extracting buffer (50 mM Tris-HCl (pH 7.5), 140 mM NaCl, 0.5% NP40, 0.25 M NDSB-201 (Calbiochem), and protease inhibitor (Complete EDTA-free, Roche)) and centrifuged at 18,500 rpm for 30 minutes to give a supernatant which in turn was reacted with an anti-KRAP antibody (J. Hum. Genet. 2007, Fujimoto, T., et al.) for 4 hours. Thereafter, the reaction mixture was reacted with protein G sepharose for another 1 hour, followed by washing the immunoprecipitated material with the above extracting buffer and boiling it with a SDS-PAGE sample buffer to give a precipitated protein.

For the investigation of the presence or absence of the intracellular localization of the KRAP protein and the IP3R1 protein, the KRAP protein with HA-tag added thereto and the IP3R1 protein with GFP-tag added thereto were transfected to HEK 293 cells and then subjected to immune cell staining.

The cells (HEK 293 cells) were incubated in DMEM (high glucose, Invitrogen) with 10% bovine serum-containing penicillin-streptomycin-glutamine mixture (Invvitrogen) at 37° C. under 5% CO₂ condition. The cells were inoculated on a collagen-coated cover glass, and a KRAP-HA expression plasmid and an IP3R1-GFP expression plasmid were introduced using lipofectamine LTX (Invitrogen). After incubation for 24 hours, the cells were fixed with 4% PFA and blocked with a blocking solution (5% bovine serum, 20 mM Tris-HCl (pH 7.5), 150 mM NaCl, and 0.1% Tx-100). Thereafter, the reaction mixture was reacted with anti-HA (3F10 clone; Roche) and anti-GFP (Clonetech) and then subjected to fluorescence staining using Alexa Fluor 488 labelled anti-rabbit antibody (Invitrogen) and Alexa Fluor 546 labelled anti-rat antibody (Invitrogen). The resulting cells (HEK 293 cells) were then observed with a confocal laser scanning microscope, and it was observed that the fluorescence signals of the both proteins coincided with each other and they were localized together with each other.

### EXAMPLE 9

A cDNA library was formed by reverse transcription using the total RNA of the mouse liver as a template and oligo-dT primers. IP3R1 coding region full length was amplified by PCR using cDNA as a template and the following primers, and it was inserted into NheI-SalI site of pEGFP-N1 vector (Clonetech) so as to align the frame with the C-terminal GFP.

Forward primer (SQ ID NO. 27):
5′-ggggctagctcaggctttccaacacggacatgtct-3′
Reverse primer (SQ ID NO. 28):
5′-ggggtcgactgggccggctgctgtgggttgacat-3′

### EXAMPLE 10

In order to examine a functional interaction between the KRAP protein and the IP3R1 protein, an ATP ligand-dependent calcium ion release from the endoplasmic reticulum was observed. Specifically, HEK 293 cells were prepared by inhibiting the expression of the KRAP gene by RNA interference, and the fluorescent signal intensity of Fluo 4 used as a calcium ion indicater was measured periodically before and after ATP stimulation.

The intracellular calcium imaging was carried out in the manner as will be described below. In HEK 293 cells were introduced 1 nM siRNA oligonucleotide by electroporation (MicroPorator MP-100; Digital Bio), and a 96-well plate was filled with DMEM having a 10% bovine serum-containing penicillin-streptomycin-glutamine mixture (high glucose; Invitrogen), and the cells were inoculated in each well at the rate of 30,000 cells per well and incubated at 37° C. under 5% CO₂ condition. At the time of 40 hours after the introduction of siRNA, 4 μM of Fluo 4 (Invitrogen) and 5 μM of Hoechst 33342 (Hoechst) were incorporated into the cells in the above culture medium at 37° C. for 50 minutes and then substituted with Hank's BSS (Ca²⁺, Mg2+-Free), followed by carrying out the intracellular calcium imaging with In Cell Analyzer 1000 (GE). ATP was injected into the cells at the final concentration of 1, 3, and 10 μM with an automatic ligand injection machine, and fluorescent pictures were periodically taken of the cells and analyzed with an analysis software (In Cell Analyzer 1000 Workstation 3.5) for quantitation. The Fluor4 fluorescence signals of the nuclear region for the total cells within a visual field were digitized, and an average per cell was computed.

The siRNA oligonucleotides used herein are the same as used in the literature document previously reported by the present inventors (J. Hum. Genet. 2007, Fujimoto T. et al.), and their amino acid sequences are as described below.

KRAP #1,
(SQ ID NO. 29)
5′-G GAG AAU GCU GAU AGU GAU AGA AUU-3′;
(SQ ID NO. 30)
3′-C CUC UUA CGA CUA UCA CUA UCU UAA-5′;
Scramble RNA #1,
(SQ ID NO. 31)
5′-G GAC GUA UAG UGU GAG AUA AAG AUU-3′;
(SQ ID NO. 32)
3′-C CUG CAU AUC ACA CUC UAU UUC UAA-5′;
KRAP #2,
(SQ ID NO. 33)
5′-C CAG CUA GGU CUU ACG AAG UCG AAA-3′;
(SQ ID NO. 34)
3′-G GUC GAU CCA GAA UGC UUC AGC UUU-5′;
Scramble RNA #2,
(SQ ID NO. 35)
5′-C CAU AGG UCU UAC GAA GUC GGC AAA-3′;
(SQ ID NO. 36)
3′-G GUA UCC AGA AUG CUU CAG CCG UUU-5′

FIG. 11 shows representative illustrations of a periodical variation in the fluorescence signals of the calcium indicator by the ATP stimulation of the cells in which the expression of the KRAP gene was inhibited and the cells in which no expression thereof was inhibited at the final ATP concentrations of 1 μM, 3 μM, and 10 μM. It was shown in FIG. 1 that the cells with the KRAP expression inhibited was weaker in fluorescence intensity at either ATP concentration than the cells treated with the scramble siRNA used as a control. In each of the figures, the upper line indicates the cells treated with the scramble siRNA, and the lower line indicates the cells in which the expression of the KRAP gene was inhibited.

FIG. 12 shows data indicating a comparison among the maximum fluorescence values after stimulation by ATP at its final concentration of 1 μM, 3 μM, and 10 μM. As is clear from FIG. 12, the maximum values were obtained in 3 or 5.5 seconds after the ATP stimulation, and a significant difference was recognized at the time of stimulation of the ATP final concentrations of 1 μM and 3 μM. The values for the cells with the KRAP expression inhibited were decreased. In the figure, the left-hand bars indicate the cells treated with the scramble siRNA, and the right-hand bars indicate the cells with the KRAP expression inhibited.

FIG. 13 is a bar graph showing mean peak amplitudes indicating ratios of the maximum fluorescence values when the value before ATP stimulation was computed as 1. A significant difference was recognized at the time of the ATP stimulation at the final concentrations of 1 μM and 3 μM, and the values for the cells with the KRAP expression inhibited were decreased. In the figure, each of the left-hand bars indicates the cells treated with the scramble siRNA, and each of the right-hand bars indicates the cells with the KRAP expression inhibited.

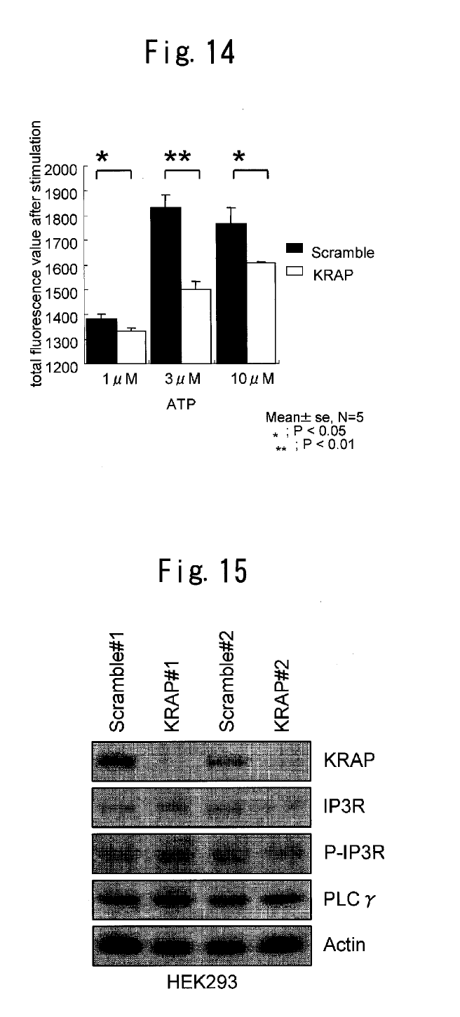

FIG. 14 is a bar graph showing total fluorescence values after stimulation indicating an addition of the fluorescence values after the ATP stimulation (from 3 seconds to 25.5 seconds after the ATP stimulation). As shown in FIG. 14, a significant decrease in the fluorescence values was recognized for the cells with the KRAP expression inhibited under all the stimulation conditions at the final ATP concentrations of 1 μM, 3 μM, and 10 μM. In the figure, each of the left-hand bars indicates the cells treated with the scramble siRNA, and each of the right-hand bars indicates the cells with the KRAP expression inhibited.

The results as described above reveal that the KRAP regulates the functions of the IP3R1 protein and it exerts an influence on the calcium ion release from the intracellular storage to the cytoplasm.

FIG. 15 is Western blots showing the inhibition of the expression of KRAP in HEK 293 cells. As is clear from FIG. 15, the inhibition of the KRAP expression did not cause any big variation in the expression amount of IP3R1 and a degree of phosphorylation of IP3R1. No change was observed of the expression amount of phospholipase C-γ located upstream of the signaling to IP3R1

As described above, the instant invention demonstrates an association of the KRAP protein and the IP3R1 protein on the basis of the experiment of immunoprecipitation using the mouse tissue and culture cells. Moreover, the physical interaction between the KRAP protein and the IP3R1 protein was also confirmed from a coincidence of their intracellular localization. Further, the measurement for the intracellular calcium kinetics established as a functional analysis of IP3R1 confirms that the inhibition of the KRAP expression negatively regulates the calcium ion release from the intracellular storage to the cytoplasm. These results indicate that the binding of the KRAP protein to the IP3R1 protein is required for demonstration of the normal functions of the IP3R1 protein.

### INDUSTRIAL APPLICABILITY

The instant invention reveals that a partial sequence of the KRAP protein, peptides corresponding thereto, or substances acting on the expression or functions of the KRAP protein exert an influence on the interaction between the KRAP protein and the IP3R protein, consequently resulting in the ability of acting as an IP3R function regulator.

It is considered that IP3R regulates one of second messengers, intracellular calcium ion, and it is working and functioning for the regulation of divergent biological phenomena including the genomic expression, cellular apoptosis, neurological functions, secretory vesicle dynamics, and so on (J. Neurochem. 2007, Mikoshiba, K.). The substances capable of upregulating or downregulating the IP3R functions may assist in providing not only a research tool for understanding various biological phenomena, but also means for developing the understanding of mechanism of disease onsets and methods for the treatment of diseases.

1.-9. (canceled)

10. Inositol 1,4,5-triphosphate receptor-binding protein in which KRAP protein (SQ ID NOS. 1-4) is bound to inositol 1,4,5-triphosphate receptor (IP3R: SQ ID NOS. 5-8).

11. Inositol 1,4,5-triphosphate receptor-binding protein as claimed in claim 10, wherein an amino acid sequence constituting the protein to be bound to IP3R is a full length sequence or an IP3R binding site-carrying region carrying an amino acid site to which IP3R is bound.

12. Inositol 1,4,5-triphosphate receptor-binding protein as claimed in claim 10, wherein said IP3R binding site-carrying region comprises an amino acid sequence region of 19 amino acids located at from amino acid 200 to amino acid 218 for mouse KRAP protein or an amino acid sequence region corresponding to that of said mouse KRAP protein for KRAP protein of a mammal other than mouse.

13. Inositol 1,4,5-triphosphate receptor-binding protein as claimed in claim 10, wherein said inositol 1,4,5-triphosphate receptor-binding protein is a structural mimic or a compound to be synthesized on the basis of information on a primary or higher amino acid conformation of said IP3R-conjugated protein or said IP3R binding site-carrying region.

14. An intercellular calcium ion concentration regulator comprising the inositol 1,4,5-triphosphate receptor-binding protein as claimed in claim 10 as a major constituent.

15. The intercellular calcium ion concentration regulator as claimed in claim 14, wherein said intercellular calcium ion concentration regulator comprises an IP3R function regulator, an IP3R activity inhibitor or an IP3R activator.

16. An assay method comprising screening a substance for inhibiting an interaction of a KRAP-IP3R protein by producing a KRAP-IP3R protein complex formed by binding of KRAP protein to IP3R protein or an IP3R binding site-carrying region and detecting a binding capacity thereof.

17. A vector comprising said inositol 1,4,5-triphosphate receptor-binding protein as claimed in claim 10 or an binding site-carrying region thereof.

18. A transgenic non-human mammalian animal carrying said inositol 1,4,5-triphosphate receptor-binding protein as claimed in claim 10 or an binding site-carrying region thereof.


SEQUENCE LISTING (36 sequences)

SEQ ID NO: 1 - DNA, 3780 bp, Human sapiens
atggaccggcccctgtcgtcgtcggcggaggcggaggaggaactggagtggcaagtggcgagtcgcaggaggaaggcctgggccaagtgccgcagctcctggcaagcgtcggagacggaggatctgtccacagaagcgacgacgcaggacgaggaggaggacgaggaggaggacctccccggcgcgcagctgccggcagcggggggaagaggaaacgtgcccaacgagaagatcgcgatatggctcaaggactgccgtacacctttgggagcctcactggatgaacaaagcagtagtacactcaagggtgtgcttgtgagaaatggaggaagttttgaagatgatttgtcattgggagctgaagccaaccacctccatgaaagtgatgctcaaattgaaaactgcaataatatcttggccaaagagagaagattacagtttcatcagaaagggagaagtatgaattccactggatctgggaaaagtagtgggacagtttcaagtgtttcagaattgttggaactttatgaggaagatcctgaagaaattctttataatcttggatttggacgtgatgaaccagatattgcttctaaaattccttccagattttttaattcatcatcctttgccaaagggatagatattaaagtatttttgagtgctcagatgcaacggatggaagtagaaaacccaaattatgctttaacaagccgttttcgtcaaattgaagtgcttactactgtggccaatgcgttttcttctttatattctcaagtctccgggacgcccctgcagagaattggaagtatgtcctcagtgacctctaacaaggagacagacccacctccacctttaactcgaagtaacactgcaaatcgtttaatgaaaacactctcaaaactgaatttatgtgttgataaaacagagaaaggagaaagtagtagtccttctccatcagctgaaaaaggaaagattctaaatgtttcagtgattgaagaaagtggcaataaaaacgatcaaaagtctcaaaaaattatgaagaagaaagagtcatcttctatgttggctacagttaaagaagaagtctctggtagttcagcagctgttacggagaatgctgatagtgatagaatttctgatgaagcaaatagtaattttaaccaaggaactgaaaatgaacaaagtaaagaaactcaaagtcatgagagtaaactgggtgaggaatctggtattgtagaatccaaattagatagtgatttcaacatatccagccacagtgagctggaaaatagcagtgagctgaaaagtgtccatatatccacacctgaaaaagagccttgtgcaccactgacaataccatccataagaaatataatgacacagcagaaggactccttcgaaatggaagaggttcaaagtacggagggagaagctcctcatgttccagccacttaccagctaggtcttacgaagtcgaaaagagatcatctgttacgtactgcaagtcagcattccgatagcagtggttttgctgaagattctacagactgcctatcccttaatcatcttcaggttcaggagtccttgcaggctatggggagtagtgctgatagttgtgacagtgagacaacagttacgtcacttggtgaagaccttgccacaccaacagcacaagaccagccttattttaatgaatcagaggaggagtctcttgtccctcttcagaagggactagagaaggcagcagcagttgcagacaaaagaaaatcaggtagccaggatttccctcagtgcaacaccattgagaatacaggaactaaacagtccacctgtagtccaggggatcatatcattgaaattactgaagtggaagaggatttgtttccagcagagacagtagagctactgagggaagcaagtgctgaaagtgatgtgggtaaaagcagtgaaagtgaatttactcagtataccacacaccatattctgaaatcattggcttctattgaagctaaatgcagtgatatgagctctgaaaatacaactgggcctccctcttccatggacagagttaatacagctttgcaaagagctcaaatgaaggtttgcagtctgtctaatcaaaggatggggcgtagcctgctaaaatcaaaagatttgttaaaacaaaggtacttatttgcaaaagctggctatcctctaagaaggtctcagtctttaccaaccaccttattgagcccagtaagggttgtgtcctctgtcaatgttcgattatctccaggaaaagagaccagatgcagcccaccttccttcacctataagtacacacctgaagaggagcaggaattggaaaagcgggtgatggaacatgatggtcagtctttagttaaatcgaccattttcatctctccatcatctgtgaagaaagaagaagccccccagagtgaggcgccgcgggtggaggaatgccatcatggaaggactcctacctgttcacggcttgctctaccaccaatgtctcagtctacctgttcccttcattccatccactctgagtggcaagaaaggcccctgtgtgagcacacaagaactctgagcactcacagtgttcccaacatatcaggggctacttgtagtgccttcgcttcccctttcgggtgtccttactcacatagacatgccacctacccttaccgagtgtgctctgtgaatcctccttcagccatagaaatgcagttgcgaagagtattacatgatattagaaactcactgcagaatctttcacagtaccctatgatgagaggacctgatcctgctgctgctccatatagtactcagaaatcatctgttctacctctttatgaaaatacttttcaggagctccaggtaatgaggcggagcctgaatttgtttagaacacaaatgatggatttagaattggcaatgctgcgtcagcaaaccatggtttatcatcatatgactgaggaggagaggtttgaagttgatcagctccagggtttgagaaattcagtccgaatggaacttcaggacctggaactgcagctggaggagcgcctgctgggcctggaggagcagcttcgtgctgtgcgcatgccttcacccttccgctcctccgcactcatgggaatgtgtggcagtagaagcgctgataacttgtcatgcccttctccattgaatgtaatggaaccagtcactgaactgatgcaggagcagtcatacctgaagtctgaattgggcctgggacttggagaaatgggatttgaaattcctcctggagaaagctcagaatctgttttttcccaagcaacatcagaatcatcttctgtatgttctggtccctctcatgctaacagaagaactggagtaccttctactgcctcagtgggcaaatccaaaaccccattagtggcaaggaagaaagtgttccgagcatcggtggctctaacgccaacagctccttctagaacaggctctgtgcagacacctccagatttggaaagttctgaggaagttgatgcagctgaaggagccccagaagttgtaggacctaaatctgaagtggaagaagggcatggaaaactcccatcaatgccagctgctgaggaaatgcataaaaatgtggagcaagatgagttgcagcaagtcatacgggagattaaagagtctattgttggggaaatcagacgggaaattgtaagtggacttttggcagcagtatcttcaagtaaagcgtctaattctaagcaagattatcattaa

SEQ ID NO: 2 - protein, 1259 aa, Human sapiens
MDRPLSSSAEAEEELEWQVASRRRKAWAKCRSSWQASETEDLSTEATTQDEEEDEEEDLPGAQLPAAGGRGNVPNEKIAIWLKDCRTPLGASLDEQSSSTLKGVLVRNGGSFEDDLSLGAEANHLHESDAQIENCNNILAKERRLQFHQKGRSMNSTGSGKSSGTVSSVSELLELYEEDPEEILYNLGFGRDEPDIASKIPSRFFNSSSFAKGIDIKVFLSAQMQRMEVENPNYALTSRFRQIEVLTTVANAFSSLYSQVSGTPLQRIGSMSSVTSNKETDPPPPLTRSNTANRLMKTLSKLNLCVDKTEKGESSSPSPSAEKGKILNVSVIEESGNKNDQKSQKIMKKKESSSMLATVKEEVSGSSAAVTENADSDRISDEANSNFNQGTENEQSKETQSHESKLGEESGIVESKLDSDFNISSHSELENSSELKSVHISTPEKEPCAPLTIPSIRNIMTQQKDSFEMEEVQSTEGEAPHVPATYQLGLTKSKRDHLLRTASQHSDSSGFAEDSTDCLSLNHLQVQESLQAMGSSADSCDSETTVTSLGEDLATPTAQDQPYFNESEEESLVPLQKGLEKAAAVADKRKSGSQDFPQCNTIENTGTKQSTCSPGDHIIEITEVEEDLFPAETVELLREASAESDVGKSSESEFTQYTTHHILKSLASIEAKCSDMSSENTTGPPSSMDRVNTALQRAQMKVCSLSNQRMGRSLLKSKDLLKQRYLFAKAGYPLRRSQSLPTTLLSPVRVVSSVNVRLSPGKETRCSPPSFTYKYTPEEEQELEKRVMEHDGQSLVKSTIFISPSSVKKEEAPQSEAPRVEECHHGRTPTCSRLALPPMSQSTCSLHSIHSEWQERPLCEHTRTLSTHSVPNISGATCSAFASPFGCPYSHRHATYPYRVCSVNPPSAIEMQLRRVLHDIRNSLQNLSQYPMMRGPDPAAAPYSTQKSSVLPLYENTFQELQVMRRSLNLFRTQMMDLELAMLRQQTMVYHHMTEEERFEVDQLQGLRNSVRMELQDLELQLEERLLGLEEQLRAVRMPSPFRSSALMGMCGSRSADNLSCPSPLNVMEPVTELMQEQSYLKSELGLGLGEMGFEIPPGESSESVFSQATSESSSVCSGPSHANRRTGVPSTASVGKSKTPLVARKKVFRASVALTPTAPSRTGSVQTPPDLESSEEVDAAEGAPEVVGPKSEVEEGHGKLPSMPAAEEMHKNVEQDELQQVIREIKESIVGEIRREIVSGLLAAVSSSKASNSKQDYH

SEQ ID NO: 3 - DNA, 3759 bp, Mus musculus
atgaaccgacccctgtcggcggaggcggaggaggaactggagtggcaagtggcgagtcgcaggaggaaggcctgggccaagtgccgcagctcctggcaggcgtcggagaccgaggatctgtccacagagacgacgacgcaggacgaggacgaggacgacgaggaggacctcccaggcacgaagctgccggcacccgcggggcgaggaaacgtgcccaacgagaagatcgcgatatggctcaaagactgccgcacccctctgggagcctcgctggatgagcaaagcagtggtacgccgaagggtgtgcttgtgagaaatggaggaagctttgaagatgacctgtcactgggagctgaagccaatcacctacatgaacctgatgctcaagttgaaaactgcaacaatatcttggccaaggagagaagactacagtttcatcagaaagggagaagtatgaattccactgggtctgggaaaagcagtggtaccgtgtcaagtgtctcagaactgctggagctttacgaggaagatcctgaagaaattctatataatcttggatttggacgagatgaaccagatattgcttctaaaattccttccagattttttaattcatcatcctttgccagaggcatagacattaaagtatttctgagtgcacagatgcaacggatggaagtagagaacccaaactatgctctgacaagccgatttcgccaaattgaagtcctgactactgtggccaatgcattttcttctctatattcccaagtctctggaactccactgcagaggattggaagtatgtcctcggtgacctccaccaaggaggtggcagactccccgccccctctaactcgaagcaacactgcaaaccgtctgatgaaaacactatcaaaactgaacttatgtgttgataaaacagagaaaggagaagggggctcttcccccgccgctgagaaaggaaggactctaagcatctcactgtctgaagatggcggcggcggcaagagtgaccctaagcttcagaaagttgtaaagaagaaggagtcgtcttcgatgctggctacggttacagaagaagtctcaggtagttcatcaactgtcacagacagcgttgatgcagacagactttccgaggaagcagacagtaccattagccaccaggaggaaagtgaagaaagcagagaggctcacagtcaggagaaggacccgctccgcaagtctgctgtgacagatcctgacttgggtcatgatggccgcgtgtccagccactgcgagctggagagcagcagtgagctgaaaagtgcccaggcgtcttcgagcgagaaagagccttgtgcacccctgacaatcccatccatacggaacataatgacgcagcagaaagactcctttgagatggaagaggtccagagcacagagggagaagcccctcacgtgccagccacttgccagctaagtcttgccaagtcaaaaagagatcatctgttgagaactgcaagtcagcattccgatagcagcggctttgctgaggactccacagactgtgtctccctcaaccacctcctggtgaacgagtccctgcaagccatggggagcagtgctgacagctgtgacagtgaaaccaccgtcacatcgcttggtgaggaccacgtgactcctacagcgcaagaccagccttacttcaatgagtcagaggaggagtccctggctcctctgcagaaaggaagagcaaaggtggaaatagtggctgagaaaaggaaagccgacaaccaagatttccctcagtgtgtgaccgcagagaatgctggaaacaatgagtccacaaagggcccgtgtgagcctggtcatcagatcacagaaacaggggagcatccacctctggcagccactggagagctccccagggaagagagtgtcgagagtgatgtagagaaaggcagtgaatgtgaatttgcccagtacaccacacaccatattctcagatccttggcttcctttgaagcgcagggaagcggtatgagctctgaaaagaaaactgggtttccctcttctgtggacagagtgaacactgccctgcagagagctcaaatgaaggtctgcagtatgtctgggcagagagtagggcgtagcctaataaaatcaaaggatctgttgaaacaaaggtacttgctcgcaaaagctggctatcctctgagaaggtctcagtctttgcccaccactttactgagcccagtacgggtggtgtcttctgtcaatgtgcgattatctccaggaaaagagaccaggtgcagcccaccgtccttcacctacaagtacacacccgaagaggaacaagacctggaaaagcaaggaactgaacacgatggtcagtctctggttaaatctaccatcttcatcccgccaccctctgtgaagaaagaagaagcccctcagagtgaggggacacgactggaggaatgccaccatggaaggcttgccccctgcccacagttcgctccaatatcccagtccacctgctcccttcactctgtccactctgagtggcaggacagacccctgtgtgaacacatgaggactctgagtgcccacagtgtccccaacatatcgggtgccgcatgtagtgccttctctcctttcgggtgtccctattcacacagacatgctgcccacccatacagggcatgctctgtgaatcctccttctgccattgagatgcagttgcggagagtgttgcatgatattagaagctcactacagaatctttcacagtatcctatgacgagaggacctgaccttgctgctgctccatacagtactcagaactcgtctgttctacctctttatgaaaataccttccaggagcttcaagtcgtgaggcggagcctgaatctgttcagaacgcagatgatggacttggagctggccatgctgcggcagcaaaccgtggtgtaccctcatatgacagaggaggacaggtatgaagttgatcagctgcagggtttgcggaactctgtccgaatggaactgcaggacctggagatgcagctggaggagcgcttgctgggcctggatgagcagctccgtgcagtgcgggtgccatcacctttccgcccctctgcactcccggggatgtgtggcagcaggagcgtagataacttatcatgcccgtctccactgaacgtcatggagccggtcacagagctaatgcgggagcaatcatacctgaagtctgagctgggcctgggccttggagacatggcgtatgaaattcctcctggagagagctcggagtccgttttctctcaagccacatcagagtcgtcttccgtctgctccagtccctcccacaccaacagaagatctagagggctacctgggagcaaacccagagcccgcttagtggcgagaaagaagatattccgagcctctgtggccctgacaccaactgccccctctagaacaggctctgtgcaaacacctccagatctggagagttccgaagaagctggaggagctgaggaagcatccccggtggtggggcttgcatctcacgtggaggaagagccagaggatctctcactgatgccagcagccgaggagatgcacaggaatgtggagcaagatgaactgcagcaagtcatccgagagattaaagagtctattgttggggaaattcgacgagaaattgtaagtggacttttggcagcagtggcttcaagtaaagcacctggtcctaagcaggacagccactga

SEQ ID NO: 4 - protein, 1252 aa, Mus musculus
MNRPLSAEAEEELEWQVASRRRKAWAKCRSSWQASETEDLSTETTTQDEDEDDEEDLPGTKLPAPAGRGNVPNEKIAIWLKDCRTPLGASLDEQSSGTPKGVLVRNGGSFEDDLSLGAEANHLHEPDAQVENCNNILAKERRLQFHQKGRSMNSTGSGKSSGTVSSVSELLELYEEDPEEILYNLGFGRDEPDIASKIPSRFFNSSSFARGIDIKVFLSAQMQRMEVENPNYALTSRFRQIEVLTTVANAFSSLYSQVSGTPLQRIGSMSSVTSTKEVADSPPPLTRSNTANRLMKTLSKLNLCVDKTEKGEGGSSPAAEKGRTLSISLSEDGGGGKSDPKLQKVVKKKESSSMLATVTEEVSGSSSTVTDSVDADRLSEEADSTISHQEESEESREAHSQEKDPLRKSAVTDPDLGHDGRVSSHCELESSSELKSAQASSSEKEPCAPLTIPSIRNIMTQQKDSFEMEEVQSTEGEAPHVPATCQLSLAKSKRDHLLRTASQHSDSSGFAEDSTDCVSLNHLLVNESLQAMGSSADSCDSETTVTSLGEDHVTPTAQDQPYFNESEEESLAPLQKGRAKVEIVAEKRKADNQDFPQCVTAENAGNNESTKGPCEPGHQITETGEHPPLAATGELPREESVESDVEKGSECEFAQYTTHHILRSLASFEAQGSGMSSEKKTGFPSSVDRVNTALQRAQMKVCSMSGQRVGRSLIKSKDLLKQRYLLAKAGYPLRRSQSLPTTLLSPVRVVSSVNVRLSPGKETRCSPPSFTYKYTPEEEQDLEKQGTEHDGQSLVKSTIFIPPPSVKKEEAPQSEGTRLEECHHGRLAPCPQFAPISQSTCSLHSVHSEWQDRPLCEHMRTLSAHSVPNISGAACSAFSPFGCPYSHRHAAHPYRACSVNPPSAIEMQLRRVLHDIRSSLQNLSQYPMTRGPDLAAAPYSTQNSSVLPLYENTFQELQVVRRSLNLFRTQMMDLELAMLRQQTVVYPHMTEEDRYEVDQLQGLRNSVRMELQDLEMQLEERLLGLDEQLRAVRVPSPFRPSALPGMCGSRSVDNLSCPSPLNVMEPVTELMREQSYLKSELGLGLGDMAYEIPPGESSESVFSQATSESSSVCSSPSHTNRRSRGLPGSKPRARLVARKKIFRASVALTPTAPSRTGSVQTPPDLESSEEAGGAEEASPVVGLASHVEEEPEDLSLMPAAEEMHRNVEQDELQQVIREIKESIVGEIRREIVSGLLAAVASSKAPGPKQDSH

SEQ ID NO: 5 - DNA, 9858 bp, Human sapiens
agtaaccatgtggatgtgctgctgaagcgtttcctcaagctcgctggggtgggaggagaggaggaggaggaggtggtggtggaggaggaggcagggggtggagagagagaaagcgcacgccgagaggaggtgtgggtgttccgcttccatcctaacggaacgagctccctcttcgcggacatgggattacccagcggctgctaacccctctcctcgccctgctcccccaaaccggcgtggctccccgggcaccaaggagctgactacagaggagcaggatttgcacccctcgctgggcttgctttggcaacagagtgcctgacccaggtcaggattttcaagaaagacatgtctgacaaaatgtctagcttcctacatattggagacatttgttctctgtacgcggagggatcgacaaatggatttattagcaccttgggcctggttgatgatcgttgtgttgtacagccagaaaccggggaccttaacaatccacctaagaaattcagagactgcctctttaagctatgtcccatgaaccgctactctgcccaaaagcagttctggaaagccgctaagcctggggccaacagcaccacagacgcagtgctactcaacaaactgcaccacgctgcagacttggaaaagaagcagaatgagacagaaaacaggaaattgctggggaccgtaatccagtatggcaatgtgatccagctcctgcatttgaaaagtaataaatacctaacagtgaataagaggcttcctgctctgttggagaagaatgccatgagagtcacattggacgaggctggaaatgaagggtcctggttttatattcagccattctacaagctgcgatccattggagacagcgtggtcataggtgacaaggtggttctgaaccccgtcaatgctggtcagcccctacatgctagcagccatcaactggtagataacccaggctgcaatgaggtcaattccgtcaactgcaatacaagctggaaaatagtccttttcatgaaatggagtgataacaaagacgacatattaaaggggggtgacgtggtgaggctgtttcatgctgagcaggagaagtttctcacctgtgacgaacacaggaagaagcagcacgtcttcctgagaaccacgggccggcagtcggccacatctgccaccagttcaaaagccctgtgggaggtggaggtggtccagcatgacccatgtcggggcggagcagggtattggaacagccttttccgtttcaagcatctggccacggggcattacttggcagcagaggtagaccctgactttgaggaagaatgcctggagtttcagccctcagtggaccctgatcaggacgcctctcgaagtaggttgcggaatgcccaagaaaagatggtatactccctggtctctgtgcctgaaggcaatgacatctcctccattttcgagctagatcccaccactctgcgtggaggtgacagccttgtcccaaggaactcttatgttcggctcagacacctatgtactaatacctgggttcacagcacaaatattcctattgacaaggaagaagaaaagcccgtgatgctgaaaattggcacctctcctgtgaaggaggataaggaagcatttgccatagttccggtttctcctgctgaagttcgggacctggactttgccaatgatgccagcaaggtgctgggctccattgctgggaagctagagaagggcaccatcacccagaatgaaaggaggtctgtaaccaagctgctagaagatttggtttacttcgtcactggtggaactaattctggtcaagatgttctcgaagttgtcttctccaagcccaacagagaacggcagaaactgatgagagaacagaatattctcaagcagatcttcaagttgttacaagccccattcacagactgcggtgatggcccaatgcttcggctggaagagctcggggaccagcggcacgctcctttcagacacatctgccggctctgctacagggtgctgagacactcgcagcaagactacaggaagaaccaggagtatatagccaagcagtttggcttcatgcagaagcagattggctatgatgtgttggctgaagacactatcactgccctgctccacaataatcggaaactcctggaaaaacacattaccgcggcagagattgacacatttgtcagcctggtgcgaaagaacagggagcccagattcttagattacctctccgacctctgtgtctccatgaacaaatcaattccagtgacccaggaactgatatgtaaagctgtgctgaaccccaccaacgctgacatcctgattgagaccaagttggttctttctcgttttgaatttgaaggtgtctcttccactggagagaatgctctggaggcaggagaagacgaggaagaggtgtggctgttttggagggacagcaacaaagagattcgcagcaagagtgtgagggaattggctcaggatgctaaagaagggcagaaggaggaccgagacgttctcagctactacagatatcagctgaacctctttgcgaggatgtgtctggaccgccaatacctggccatcaacgaaatctcaggccagctggatgtcgatctcattctccgctgcatgtctgacgagaacctgccctatgacctcagggcgtccttctgccgcctcatgcttcacatgcatgtggaccgagatccccaggaacaagtcacccccgtgaaatatgcccgcctctggtcggagattccctcggagatcgccattgacgactatgatagtagtggagcttccaaagatgaaattaaggagagatttgctcagaccatggagtttgtggaggagtatttaagagatgtggtttgtcagaggttccctttctctgataaagagaagaataagcttacgtttgaggttgtaaatttagctaggaatctcatatactttggtttctacaacttctctgaccttctacgattaactaagatccttctggccatattggactgtgtacatgtgacaacaatcttccccattagcaagatggcgaaaggagaagagaataaaggcagtaacgtgatgagatctattcatggcgtgggagagctgatgacccaggtggtgctccggggaggaggctttttgcccatgactcccatggctgctgcccctgaaggcaatgtgaagcaggcagagcctgagaaggaggacatcatggtcatggacaccaagctgaagatcattgagatactccagtttattttgaatgtgaggttggattataggatctcctgcctcctgtgtatatttaagcgagagtttgatgaaagcaattcccagacttcagaaacatcctccggaaacagcagccaagaagggccaagtaatgtaccaggtgctcttgactttgaacacattgaagaacaagcagaaggcatctttggaggaaggaaagagaacaccccactggacttggatgaccacggcggcagaacctttctccgtgtcctgctccacttgacgatgcatgactacccacccctggtgtcaggggccctgcagctcctcttccggcacttcagccagaggcaggaggtgctccaggccttcaaacaggttcaactgctggttaccagccaagatgtggacaactacaaacagatcaaacaagacttggatcaactgaggtccatcgtggaaaagtcagagctttgggtgtacaaagggcagggccccgatgagactatggatggtgcatctggagaaaatgaacataagaaaacggaggagggaaataacaagccacaaaagcatgaaagcaccagcagctacaactacagagtggtcaaagagattttgattcggcttagcaaactctgtgttcaagagagtgcctcagtgagaaagagcaggaagcagcaacagcgtctgctccggaacatgggcgcgcacgccgtggtgctggagctgctgcagattccctatgagaaggccgaagataccaagatgcaagagataatgaggttggctcatgaatttttgcagaatttctgcgcaggcaaccagcagaatcaagctttgctacataaacacataaacctgtttctcaacccagggatcctggaggcagtaaccatgcagcacatcttcatgaacaatttccagctttgcagtgagatcaacgagagagttgttcagcacttcgttcactgcatagagactcacggtcggaatgtccagtatataaagttcttacagacaattgtcaaggcagaagggaaatttattaaaaaatgccaagacatggttatggccgagctggtcaattcgggagaggatgtcctcgtgttctacaacgacagagcctctttccagactctgatccagatgatgcggtcagaacgggatcggatggatgagaacagccctctcatgtaccacatccacttggtcgagctcctggctgtgtgcacggagggtaagaatgtctacacagagatcaagtgcaactccctgctcccgctggatgacatcgttcgcgtggtgacccacgaggactgcatccctgaggttaaaattgcatacattaacttcctgaatcactgctatgtggatacagaggtggaaatgaaggagatttataccagcaatcacatgtggaaattgtttgagaatttccttgtagacatctgcagggcctgtaacaacactagtgacaggaaacatgcagactcgattttggagaagtatgtcaccgaaatcgtcatgagtattgttactactttcttcagctctcccttctcagaccagagtacgactttgcagactcgccagcctgtctttgtgcaactgctgcaaggcgtgttcagggtttaccactgcaactggttaatgccaagccaaaaagcctccgtggagagctgtattcgggtgctgtctgatgtagccaagagccgggccattgccattcccgtggacctggacagccaagtcaacaacctctttctcaagtcccacagcattgtgcagaaaacagccatgaactggcggctctcagcccgcaatgccgcacgcagggactctgttctggcagcttccagagactaccggaatatcattgagagattgcaggacatcgtctccgcgctggaggaccgtctcaggcccctggtgcaggcagagttatctgtgctcgtggatgttctccacagacccgagctgcttttcccagagaacacagacgccagaaggaaatgtgaaagtggcggtttcatttgcaagttaataaagcatacaaaacagctgctagaagaaaatgaagagaagctctgcattaaggtcctacagaccctgagggaaatgatgaccaaagatagaggctatggagaaaagggtgaggcgctcaggcaagttctggtcaaccgttactatggaaacgtcagaccttcgggacgaagagagagccttaccagctttggcaatggcccactgtcagcaggaggacccggcaagcccgggggaggagggggaggttccggatccagctctatgagcaggggtgagatgagtctggccgaggttcagtgtcaccttgacaaggagggggcttccaatctagttatcgacctcatcatgaacgcatccagtgaccgagtgttccatgaaagcattctcctggccattgcccttctggaaggaggcaacaccaccatccagcactcctttttctgtcgcttgacagaagataagaagtcagagaaattctttaaggtgttttatgaccggatgaaggtggcccagcaagaaatcaaagcaacagtgacagtgaacaccagtgacttgggaaataaaaagaaagacgatgaggtagacagggatgccccatcacggaaaaaagctaaagagcccacaacacagataacagaagaggtccgggatcagctcctggaggcctccgctgccaccaggaaagccttcaccactttcaggagggaggctgatcccgacgaccactaccagcctggagagggcacccaggccactgccgacaaggccaaggacgacctggagatgagcgcggtcatcaccatcatgcagcccatcctccgcttccttcagctcctgtgtgaaaaccacaaccgagacctgcagaacttcctccgttgccaaaataacaagaccaactacaatttggtatgtgagaccctgcagtttctggactgtatttgtggaagcacaactggaggccttggtcttctgggcttgtatataaatgaaaagaacgtagcgcttatcaaccaaaccctggaaagtctgaccgaatactgtcaaggaccttgccatgagaaccagaactgcatagccacccatgaatccaatggcattgacatcatcacagccctgatcctcaatgatatcaatcctttgggaaagaagaggatggaccttgtgttagaactgaagaacaatgcctcgaagttgctcctggccatcatggaaagcaggcacgacagtgaaaacgcagagaggatactttataacatgaggcccaaggaactggtggaagtgatcaagaaagcctacatgcaaggtgaagtggaatttgaggatggagaaaacggtgaggatggggcggcgtcccccaggaacgtggggcacaacatctacatattagcccatcagttggctcggcataacaaagaacttcagagcatgctgaaacctggtggccaagtggacggagatgaagccctggagttttatgccaagcacacggcgcagatagagattgtcagattagaccgaacaatggaacagatagtctttcccgtgcccagcatatgtgaattcctaaccaaggagtcaaaactacgaatttactatactacagagagagacgaacaaggcagcaaaatcaatgatttctttctgcggtctgaagacctcttcaatgaaatgaattggcagaagaaactgagagcccagcccgtgttgtactggtgtgcccgcaacatgtctttctggagcagcatttcgtttaacctggccgtcctgatgaacctgctggtggcgtttttctacccgtttaagggagtccgaggaggaaccctggagccccactggtcgggactcctgtggacagccatgctcatctctctggccatcgtcattgccctccccaagccccatggcatccgggccttaattgcctccacaattctacgactgatattttcagtcgggttacaacccacgttgtttcttctgggcgctttcaatgtatgcaataaaatcatctttctaatgagctttgtgggcaactgtgggacattcacaagaggctaccgagccatggttctggatgttgagttcctctatcatttgttgtatctggtgatctgtgccatggggctctttgtccatgaattcttctacagtctgctgctttttgatttagtgtacagagaagagactttgcttaatgtcattaaaagtgtcactcgcaatggacggtccatcatcctgacagcagttctggctctgatcctcgtttacctgttctcaatagtgggctatcttttcttcaaggatgactttatcttggaagtagataggctgcccaatgaaacagctgttccagaaaccggcgagagtttggcaagcgagttcctgttctccgatgtgtgtagggtggagagtggggagaactgctcctctcctgcacccagagaagagctggtccctgcagaagagacggaacaggataaagagcacacatgtgagacgctgctgatgtgcattgtcactgtgctgagtcacgggctgcggagcgggggtggagtaggagatgtactcaggaagccgtccaaagaggaacccctgtttgctgctagagttatttatgacctcttgttcttcttcatggtcatcatcattgttcttaacctgatttttggggttatcattgacacttttgctgacctgaggagtgagaagcagaagaaggaagagatcttgaagaccacgtgctttatctgtggcttggaaagagacaagtttgacaacaagactgtcacctttgaagagcacatcaaggaagaacacaacatgtggcactatctgtgcttcatcgtcctggtgaaagtaaaggactccaccgaatatactgggcctgagagttacgtggcagaaatgatcaaggaaagaaaccttgactggttccccaggatgagagccatgtcattggtcagcagtgattctgaaggagaacagaatgagctgagaaacctgcaggagaagctggagtccaccatgaaacttgtcacgaacctttctggccagctgtcggaattaaaggatcagatgacagaacaaaggaagcagaaacaaagaattggtcttctaggacatcctcctcacatgaatgtcaacccacaacaaccagcataagcaaatgaaagaaaggaattgtatttaccttttataattattattagtgtgggtatggctaatgagttctgattcacccacgaaggttacatttatgctgaatacatttgtaaatactcagttttatactgtatgtatatgattgctactctaaaggtttggatatatgtattgtaattagaattgttggcatgatgacatttcatttgtgccaaaaatattaaaaatgccttttttggaaggactaacagaaagcacctgatttgcacttgaaccagattatagatttaaaagtatatgacatgtattttgtatttaaaactagaatagccagtatttatgttttttataaaactgtgcaatacgaattatgcaatcacaatacatttgtagctcccgagtgtcctaaagggagtgcacttctttgaagctggtgtgttaatactatgtaataaatggttaactttcaaatgatgctgctgccaaaattatattaatagtgagtttcaggcccctgggcattttgtaccatgtaattatcctctggtgatgctgtttctcgttagtggcagtagtgcctccgtctcctagtgataatgctccaagtctatgaactgttaaatcagcattcattttaagaaaagcaactttagtttcaaagatacttttaagcttctaaattgatcatttaaactatttctttaaataagagagccaaattagaggctcatactttagcttgtgaagaagataatgaattttttaaagggaactttctatgcaatgttcaggataaatgcatactgctggccaatcagtgtcatctcctgggtaaattttgatgtcgcattataaagacatgcataattgatggtttctagattatctagtccaaacaatagagtttattttttcttcatctgaaccaacatgctacagtagctaagaagtattaaaactatatacatccatataaagatgaaatatgaactatctcattagaagtcatagttgaccacagacatgttattcttctgaaagagccacattttggttttatttcttgtcacatgatttcttttcttgatggatgaaaaatatgaaaggaaacttttatatctgttgcctagttttgtacatggatctcattttacaagagaatctctctgcaaaaaaaaaaaaaacagtttaaaaatgcattgaaagcagagttctgaaatgagtaaagtttgtaaatgcatatataaaaatatttaataaatgatgcagaatatacagtgactggttggtggctttcatttggcatttgtgacttaactgctattccatttatgtactttctttaggatcagtttgaagtacagtcggtttgattac

SEQ ID NO: 6 - protein, 2710 aa, Human sapiens
MSDKMSSFLHIGDICSLYAEGSTNGFISTLGLVDDRCVVQPETGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPVKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMAKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQTSETSSGNSSQEGPSNVPGALDFEHIEEQAEGIFGGRKENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDETMDGASGENEHKKTEEGNNKPQKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSILEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHSIVQKTAMNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKGEALRQVLVNRYYGNVRPSGRRESLTSFGNGPLSAGGPGKPGGGGGGSGSSSMSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQPGEGTQATADKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQSMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLVICAMGLFVHEFFYSLLLFDLVYREETLLNVIKSVTRNGRSIILTAVLALILVYLFSIVGYLFFKDDFILEVDRLPNETAVPETGESLASEFLFSDVCRVESGENCSSPAPREELVPAEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIKERNLDWFPRMRAMSLVSSDSEGEQNELRNLQEKLESTMKLVTNLSGQLSELKDQMTEQRKQKQRIGLLGHPPHMNVNPQQPA

SEQ ID NO: 7 - DNA, 9873 bp, Mus musculus
gcagtaaccatgtggatgttctgctgaagcgtttcctcaagcctgccggggtgggaggagaggaggaggtggtggtggtggaggaggtggaggcagagggtggagagagagaaagcgcacgccgagaggaggtgtgggtgttccgctcccatcctaacggaacgagctccctcttcgcggacatgggattgcccagcggctgctaacccctctcctggtcctgatcccccaaaccggcgtggctccccggtcaccaaggagctgattacaagggaccaggatttgcatccttggctgggcgtccattggctacagagtgcctgacctgggtcaggctttccaacacggacatgtctgacaaaatgtcgagtttcctacatattggagacatttgttctctgtatgcggagggatctacgaatggatttatcagcaccttaggcttggttgatgaccgttgtgttgtacagccagaagccggggaccttaacaatccacccaagaaattcagagactgcctctttaagctatgtcctatgaatcgatactccgcacagaaacagttctggaaagctgctaagcccggggccaacagcactacagatgcagtgctgctcaacaaattgcatcatgctgcagacttggaaaagaagcagaatgagacagaaaacaggaaattgttggggaccgtcatccaatatggcaacgtgatccagctcctgcatttgaaaagcaataaatacctgactgtgaataagaggctcccagccttgctagagaagaacgccatgagggtgacgttggacgaggctggaaatgaagggtcctggttttacattcaaccattctacaagcttcgctccatcggagacagtgtggtcataggcgacaaggtagttttgaatcctgtcaatgctggccagcctctacatgccagcagtcatcagctggtggataacccaggctgcaatgaggtcaactccgtcaactgtaatacaagctggaagatagtgcttttcatgaaatggagtgataacaaagacgatattctcaaaggaggtgatgtggtgaggctcttccatgccgagcaagagaagtttctcacctgcgatgagcaccggaagaagcagcatgtgttcctgaggaccaccggcaggcagtcagccacatcggccaccagttccaaagccctgtgggaagtggaggtagtccagcacgacccatgtcggggtggagctgggtactggaatagcctcttccggttcaagcacctggctacagggcattacttggctgcagaggtagaccctgactttgaggaagaatgcctggagtttcagccctcagtggaccctgatcaggatgcatctcggagtaggttgagaaacgcgcaagaaaaaatggtatactctctggtctccgtgcctgaaggcaacgacatctcctccatctttgagctagaccccacgactctgcgtggaggtgacagccttgtcccaaggaactcctatgtccgtctcagacacctgtgcaccaacacctgggtacacagcacaaacatccccatcgacaaggaagaggagaagcctgtgatgctgaaaattggtacctctcccctgaaggaggacaaggaagcatttgccatagttcctgtttcccctgctgaggttcgggacctggactttgccaatgatgccagcaaggtgctgggctccatcgctgggaagttggaaaagggcacaatcacccagaatgagagaaggtctgtcacgaagcttttggaagacttggtttactttgtcacgggtggaactaactctggccaagacgtgcttgaagttgtcttctctaagcccaatcgagagcggcagaagctgatgagggaacagaatattctcaagcagatcttcaagctgttgcaggcccccttcacggactgcggggatggcccgatgcttcggctggaggagctgggggatcagcgccatgctcctttcagacatatttgccgactctgctacagggtcctgcgacactcacagcaagactacaggaagaaccaggagtacatagccaagcagtttggcttcatgcagaagcagattggctatgacgtgctggccgaagacaccatcactgccctgctccacaacaaccggaaactcctggagaagcacatcaccgcggcagagattgacacgtttgtcagcctggtgcgaaagaacagggagcccaggttcttggattacctctctgacctctgcgtatccatgaacaagtcaatccctgtgacacaggagctcatctgtaaagctgtgctcaatcccaccaatgctgacatcctgattgagaccaagctggttctttctcgttttgagtttgaaggcgtttccactggagagaatgctctggaagccggggaggatgaggaagaggtgtggctgttctggagggacagcaacaaagagatccgtagtaagagtgtccgggaattggcgcaagatgctaaagagggacagaaggaagacagggacatcctcagctactacagatatcagctgaacctctttgcaaggatgtgtctggaccgccagtacctggccatcaatgaaatctccgggcagctggatgttgatctcattctccgctgcatgtctgacgagaacctcccctacgacctcagggcatccttttgccgcctcatgcttcacatgcatgtggaccgagatccccaagagcaggtgacacctgtgaaatatgcccgactgtggtcagaaattccctctgagatcgccattgatgactatgacagcagtggaacatccaaagatgaaattaaggagaggtttgcacagacgatggagtttgtggaggagtacctaagagatgtggtttgtcaaagattccccttctctgataaggagaaaaataagctcacgtttgaggttgtgaacttagccaggaatctcatatactttggtttctacaacttttctgaccttctccgattaaccaagatcctcttggcaatcttagactgtgtccatgtgaccactatcttccccattagcaagatgacaaaaggagaagagaataaaggcagtaacgtgatgaggtctatccatggcgttggggagctgatgacccaggtggtgctgcggggaggaggcttcttgcccatgactcccatggctgcggcccctgaaggaaatgtgaagcaggcagagccagagaaagaggacatcatggtcatggacaccaagttgaagatcattgaaatactccagtttattttgaatgtgagattggattataggatctcctgcctcctgtgtatatttaagcgagagtttgatgaaagcaattcccagtcatcagaaacatcctccggaaacagcagccaggaagggccaagtaatgtgccaggtgctcttgactttgaacacattgaagaacaagcggaaggcatctttggaggaaggaaagagaacacacctttggacctggatgaccatggtggcagaaccttcctcagggtcctgctccacttgacaatgcatgactacccacccctggtgtctggggccctgcagctcctctttcggcacttcagccagaggcaggaggtccttcaggccttcaaacaggttcaactgctggttactagccaagatgtggacaactacaaacagatcaagcaagacttggaccaactaaggtccattgtggagaagtctgagctctgggtgtacaaaggccaaggtcccgatgagcctatggacggagcctccggtgaaaatgagcataagaaaaccgaggaggggacgagcaagccactgaagcacgagagcaccagcagctacaactaccgagtggtgaaagagattttgattcgacttagcaagctctgcgtgcaggagagcgcgtcggtgaggaagagccggaagcagcagcaacgactgctgaggaacatgggcgcacacgctgtggtgctggagctgctgcagatcccctacgagaaggccgaagacacaaagatgcaagagatcatgcggctggctcatgaatttttgcagaatttctgtgcaggcaaccagcagaatcaagctttgctgcataaacacataaacctgtttctcaacccagggatcctggaggcagtgacgatgcagcacatcttcatgaacaacttccagctgtgcagtgagatcaacgagagagtggtccagcactttgttcactgcatagagacccacggtcgaaacgtccagtatatcaagtttctccagacgattgtcaaggcagaagggaaattcattaaaaagtgccaagacatggtcatggctgagcttgtcaactctggagaggacgtcctcgtgttctacaatgacagagcctctttccagactctgatccagatgatgcggtccgagcgtgaccggatggatgagaacagccctctcatgtaccacatccatctggtggagctcttggccgtgtgcacagagggcaagaatgtgtacacggagatcaagtgcaactccttgctcccgctcgatgacatcgttcgtgtggtcactcatgaagactgcatccccgaggttaagatcgcttacattaacttcctgaatcactgctatgtggatacggaggtggagatgaaggagatttacacaagcaaccacatgtggaagttgtttgagaatttcctcgtggacatctgcagggcctgtaacaacacaagcgacaggaagcacgcagactccattctggagaagtacgtcactgaaatcgtgatgagcatcgtcaccaccttcttcagctctcccttctcagaccagagcaccactctgcagacccgccagcctgtctttgtgcaactcctgcaaggcgtgttccgagtttaccactgcaactggctgatgccgagccaaaaagcctcggtggagagctgcatccgggtgctctctgacgtagccaagagccgggccatagccattcctgttgacctggacagccaagtcaacaacctcttcctgaagtcccacaacattgtgcagaaaacagccctgaactggcggttatcagcccgaaacgccgctcgcagagactctgtactggcagcatccagagactaccgaaatatcattgagaggttacaggacatcgtgtctgccctagaggaccggctcaggcccctggtgcaggctgagctgtctgtgctcgtggatgttctacacagaccagaactgctcttccccgagaacacggatgccaggaggaaatgtgagagtggaggtttcatctgcaagctaataaaacataccaagcaactgctggaggagaatgaagagaaactatgcattaaagtcttacagaccctcagggaaatgatgaccaaagacagaggctatggagagaagcaaatttccattgatgaatcggaaaatgccgagctgccacaggcaccggaagctgagaactccacagagcaggagcttgaaccaagtccacccctgaggcaactggaagaccataaaaggggtgaggcactccgacaaattttggtcaaccgttactatggaaacatcagaccttcaggaagaagagagagccttaccagctttggcaatggcccactatcaccaggaggacccagcaagcctggtggaggagggggaggtcctggatctagttccacaagcaggggtgagatgagcctggctgaggttcagtgtcacctcgacaaggagggggcctccaacctggtcatcgatctcataatgaatgcatccagtgaccgagtattccatgaaagcattctgctggccatcgcacttctggaaggaggcaacaccaccatccagcactcgtttttctgccggctgacagaagataagaaatcagagaagttcttcaaggttttttacgatcgaatgaaggtggcccagcaggaaatcaaggcgacagtgacagtgaacaccagcgacttgggaaacaaaaagaaagatgatgaagtggacagggatgccccgtctcggaagaaagccaaagagcccacaacacagataacagaagaggtccgggatcagctcctggaagcatctgctgccaccaggaaagcctttaccaccttccggagggaggccgaccctgatgaccattaccagtctggggagggcacccaggctacaaccgacaaagccaaggatgacctagagatgagcgctgtcatcaccatcatgcagcctatcctgcgcttcctgcagctgctgtgtgaaaaccacaaccgagatctgcagaatttccttcgttgccaaaataataagaccaactacaatttggtgtgtgagacactgcagtttctggactgtatttgtgggagcacaaccggaggccttggtcttcttggactgtacataaatgaaaagaatgtagcacttatcaaccaaaccctggagagtctgacggagtactgtcaagggccttgccatgagaaccagaactgcatcgccacccacgagtccaatggcatcgatatcatcacagccctcatcctcaatgatatcaaccctctgggaaagaagcggatggacctggtgttagaactgaagaacaatgcttcgaagctgctactggccatcatggaaagcagacacgatagtgaaaatgcagagaggatcctgtacaacatgaggcccaaggagctggtggaagtgatcaagaaggcctacatgcaaggtgaagtggaatttgaggatggggagaacggtgaggatggagctgcctcacccaggaacgtgggccacaacatctacatcctcgctcaccagttggctcggcataacaaagaacttcaaaccatgctgaaacctggaggccaggtggatggggatgaagctctggagttctacgcgaagcacacagcacaaattgagattgtcagactggaccggacaatggaacagatcgtcttccctgtgcccagcatctgtgaattcctgactaaggaatcgaaacttcgaatatattacaccacagagcgggatgagcaaggtagcaagatcaatgacttcttcctgcgctccgaggacctctttaacgagatgaactggcagaagaaacttcgagcccagcctgtcttgtactggtgtgcccgaaacatgtctttctggagcagcatctccttcaacctggccgtcctgatgaacctgctggtggcgtttttctatccatttaaaggagtaaggggaggaacactagagccacactggtcaggccttctgtggacagccatgctcatctctctggccattgtcattgctctgcccaagccccacggcatccgggccttaattgcttctacaatcctacgactgatattttcagttgggttgcagcccacactgtttctgctgggagctttcaatgtctgcaataaaatcatcttcctgatgagctttgtgggcaactgtgggaccttcaccagaggctaccgggccatggttctggatgtggagttcctctatcatttgctgtatctactcatctgtgccatgggcctcttcgtacatgagttcttctatagcttgctgctttttgatttagtgtacagagaggagactttgcttaatgtcattaaaagtgtcacccgcaatggacggtccatcatcttgacagcggtcctggctctgatcctggtttacctgttctcaattgtgggctatctgttcttcaaggatgactttatcttggaagtagataggttgcccaatgaaacagctgttccagaaactggcgagagtttggccaacgatttcctgtactctgatgtgtgcagggtagagacgggggagaactgcacctctcctgcacccaaagaagagctgctccctgccgaagaaacggaacaggataaggaacacacgtgtgagaccctgctcatgtgcatcgtcactgttctgagtcacgggctgcggagtgggggaggggtaggagacgtgctcaggaagccatccaaagaggagcctctgtttgctgcaagggtgatctacgacctcctcttcttcttcatggtcatcatcatcgtcctgaacctgattttcggggtcatcattgacacctttgctgacctgaggagtgagaagcaaaagaaggaggagatcttaaaaaccacgtgcttcatctgcggcttggaaagggacaagtttgacaataagactgtcacctttgaagagcacatcaaggaagagcacaacatgtggcactatctgtgcttcatcgtgctggtgaaagtgaaggactccacagagtacaccgggcctgagagttacgtggcagagatgatcagggaaagaaaccttgattggttccccagaatgagagccatgtccctggtcagcagcgattctgaaggggaacagaacgagctgaggaacctgcaggagaagctggagtctaccatgaagctggtcaccaatctttctggccagctgtcagaactaaaggaccagatgacagaacagaggaagcagaaacaaagaatcggccttctaggacatcctcctcacatgaatgtcaacccacagcagccggcctaggcaaatgaggcagagggactctgctcagccctctgtatatcactgtcagggtgggtacggctcattggttctgatttgcccactaagggtacatgtgcgcttagtacatttgtaaatactcagttttgtattgtatgtatatgattgctattctcagaggtttggactttcgtattgtaattagctctgttggcatggtgacttgtcactcctgccaaaaatattaaaaatgccttttttggaaggactacagaaagtacctgatttgcacttgaaccagattatagatttaaaagtatatgacatgtattttgtatttaaaactagaatagccagtatttatgttttttataaaactgtgcaatacaaattatgcaatcaccataactttgtaactcctgagtgtcctaagggagtacacatctttgaagctgatttgttgatactcgtgtaataaatggttaaatatcaaatgctgctgctgctgccaaaattatattaatagcgagtttctggcccctgggcaattttgtaccttgtaattatcctatggtgatgctgtttctcgttgctaatggcattagtgcccctgtatcctagtgataactccaggtctgtgaaccattcaaacagcattcattttgagaaaagcaactttagtttcaaggataattttaagcttcaaaattaatcatttaaagtgtttctttaagagagccatgttagaggctcacactttagcttgaaaggagttgatgaattaattttttaaagggaactttttacatgacgtttggaataacagcatattgctgaccagtcagtgtcatctcccgggtgaattttgatgtcacgttatagtcaaatgagttagctgatggtttctagattttcttcctctgaaccatgatgcagtaggtaagaagttattatgcgtatatacatatatacattcatatacgacaaagtaggagctgtccccttaggatgcatagctgcccctagggtacgtagctgaacactgacaatggcgttcttctgaaagagccacgtttgggttttatttctttgtcacatgatttcttttctggatgggtgcaaagtatcacaggaagtgttttctctctgtcgccttgttttgtacctgggtctcgctttactagaccgtctctgcacaaaagtttaaaaactgaaccgtatgcagagttccgaagcaagtcaagtttgtaaatgcatacctaaaaatatttaataaacgatgcagaatcctaaa

SEQ ID NO: 8 - protein, 2749 aa, Mus musculus
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDILSYYRYQLNLFARMCLDRQYLAINEISGQLDVDLILRCMSDENLPYDLRASFCRLMLHMHVDRDPQEQVTPVKYARLWSEIPSEIAIDDYDSSGTSKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGRKENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSILEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTALNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDESENAELPQAPEAENSTEQELEPSPPLRQLEDHKRGEALRQILVNRYYGNIRPSGRRESLTSFGNGPLSPGGPSKPGGGGGGPGSSSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRSIILTAVLALILVYLFSIVGYLFFKDDFILEVDRLPNETAVPETGESLANDFLYSDVCRVETGENCTSPAPKEELLPAEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFPRMRAMSLVSSDSEGEQNELRNLQEKLESTMKLVTNLSGQLSELKDQMTEQRKQKQRIGLLGHPPHMNVNPQQPA

SEQ ID NO: 9 - DNA, 37 bp, Artificial Sequence
  Oligonucleotide primer designed to amplify the full length coadin g mouse KRAP
gggctcgagcggcgcggccatgaaccgacccctgtcg

SEQ ID NO: 10 - DNA, 60 bp, Artificial Sequence
  Oligonucleotide primer designed to amplify the full length coadin g mouse KRAP
gggggatcctcaagcgtaatctggaacatcgtatgggtagtggctgtcctgcttaggacc

SEQ ID NO: 11 - DNA, 22 bp, Artificial Sequence
  Oligonucleotide primerdesigned to amplify the full length coading mouse KRAP insert
caccatgaaccgacccctgtcg

SEQ ID NO: 12 - DNA, 60 bp, Artificial Sequence
  Oligonucleotideprimer designed to amplify the full length coading mouse KRAP insert
ctactaagatctctaagcgtagtctgggacgtcgtatgggtagtggctgtcctgcttagg

SEQ ID NO: 13 - DNA, 30 bp, Artificial Sequence
  Oligonucleotide primer designed to amplify the mouse KRAP F202A mutant
gcatttaattcatcatcctttgccagaggc

SEQ ID NO: 14 - DNA, 29 bp, Artificial Sequence
  Oligonucleotide primer designed to amplify the mouse KRAP F202A mutant
tctggaaggaattttagaagcaatatctg

SEQ ID NO: 15 - DNA, 27 bp, Artificial Sequence
  Oligonucleotideprimer designed to amplify the mouse KRAP F203A mutant
gcaaattcatcatcctttgccagaggc

SEQ ID NO: 16 - DNA, 27 bp, Artificial Sequence
  Oligonucleotideprimer designed to amplify the mouse KRAP F203A mutant
aaatctggaaggaattttagaagcaat

SEQ ID NO: 17 - DNA, 30 bp, Artificial Sequence
  Oligonucleotide primer designed to amplify the mouse KRAP F202A/F 203A mutant
gcagcaaattcatcatcctttgccagaggc

SEQ ID NO: 18 - DNA, 29 bp, Artificial Sequence
  Oligonucleotide primer designed to amplify the mouse KRAP F202A/F 203A mutant
tctggaaggaattttagaagcaatatctg

SEQ ID NO: 19 - protein, 19 aa, Homo sapiens
  human KRAP 202-220
SRFFNSSSFAKGIDIKVFL

SEQ ID NO: 20 - protein, 19 aa, Mus musculus
  mouse KRAP 200-218
SRFFNSSSFARGIDIKVFL

SEQ ID NO: 21 - protein, 19 aa, Mouse
  mouse KRAP F202A/F203A mutant
SRAANSSSFARGIDIKVFL

SEQ ID NO: 22 - protein, 19 aa, Mouse
  mouse KRAP F202A mutant
SRAFNSSSFARGIDIKVFL

SEQ ID NO: 23 - protein, 19 aa, Mouse
  mouse KRAP F203A mutant
SRFANSSSFARGIDIKVFL

SEQ ID NO: 24 - DNA, 20 bp, Artificial Sequence
  Oligonucleotide primer designed to amplify the mouse genomic DNA
gtcacagagctaatgcggga

SEQ ID NO: 25 - DNA, 20 bp, Artificial Sequence
  Oligonucleotide primer designed to amplify the mouse genomic DNA
cttcacactgcagttgagtc

SEQ ID NO: 26 - DNA, 19 bp, Artificial Sequence
  Oligonucleotide primer designed to amplify the mouse genomic DNA
ggcttcatgatgtccccat

SEQ ID NO: 27 - DNA, 35 bp, Artificial Sequence
  Oligonucleotide primer designed to amplify the full length coding mouse IP3R1
ggggctagctcaggctttccaacacggacatgtct

SEQ ID NO: 28 - DNA, 34 bp, Artificial Sequence
  Oligonucleotide primer designed to amplify the full length coding mouse IP3R1
ggggtcgactgggccggctgctgtgggttgacat

SEQ ID NO: 29 - RNA, 25 nt, Artificial Sequence
  siRNA oligonucleotide to amplify KRAP #1 for introduction into HE K 293 cells
ggagaaugcugauagugauagaauu

SEQ ID NO: 30 - RNA, 25 nt, Artificial Sequence
  siRNA oligonucleotide to amplify KRAP #1 for introduction into HE K 293 cells
aauucuaucacuaucagcauucucc

SEQ ID NO: 31 - RNA, 25 nt, Artificial Sequence
  siRNA oligonucleotide to amplify scramble RNA #1 for introduction into HEK 293 cells
ggacguauagugugagauaaagauu

SEQ ID NO: 32 - RNA, 25 nt, Artificial Sequence
  siRNA oligonucleotide to amplify scramble RNA #1 for introduction into HEK 293 cells
aaucuuuaucucacacuauacgucc

SEQ ID NO: 33 - RNA, 25 nt, Artificial Sequence
  siRNA oligonucleotide to amplify KRAP #2 for introduction into HE K 293 cells
ccagcuaggucuuacgaagucgaaa

SEQ ID NO: 34 - RNA, 25 nt, Artificial Sequence
  siRNA oligonucleotide to amplify KRAP #2 for introduction into HE K 293 cells
uuucgacuucguaagaccuagcugg

SEQ ID NO: 35 - RNA, 25 nt, Artificial Sequence
  siRNA oligonucleotide to amplify scramble RNA #2 for introduction into HEK 293 cells
ccauaggucuuacgaagucggcaaa

SEQ ID NO: 36 - RNA, 25 nt, Artificial Sequence
  siRNA oligonucleotide to amplify scramble RNA #2 for introduction into HEK 293 cells
uuugccgacuucguaagaccuaugg